(19) United States
(12) Patent Application Publication
(10) Pub. No.: US 2015/0083906 A1
(43) Pub. Date: Mar. 26, 2015

(54) BIOMARKERS FOR MONITORING INTERVENTION THERAPIES FOR DIABETES

(71) Applicant: MERCK SHARP & DOHME CORP., Rahway, NJ (US)

(72) Inventors: Huseyin Mehmet, Hoboken, NJ (US); Sandra C. Souza, Jersey City, NJ (US); Hidehisa Asada, Washington Township, NJ (US); Yoshiaki Miura, Glen Rock, NJ (US); Taku Nakahara, West New York, NJ (US); Diane McCarthy, Warrington, PA (US)

(21) Appl. No.: 14/394,872

(22) PCT Filed: Apr. 16, 2013
(86) PCT No.: PCT/US13/36690

§ 371 Date: Oct. 16, 2014

Related U.S. Application Data
(60) Provisional application No. 61/635,971, filed on Apr. 20, 2012.

(51) Int. Cl. G01N 33/96; H01J 49/16; H01J 49/34

(52) U.S. Cl. CPC G01N 33/96; H01J 49/34; H01J 49/164
USPC 250/282

(57) ABSTRACT

The use of N-linked glycosylation pattern of serum proteins as a biomarker for evaluating the efficacy of intervention therapies for diabetes is disclosed. As disclosed herein, changes in the N-linked glycosylation of total plasma proteins precedes and predicts the decrease in glycated hemoglobin (HbA1c) associated with successful treatment of diabetes. Therefore, measuring changes in N-linked glycosylation of total serum plasma proteins over time may be used as a biomarker to evaluate or access the efficacy of an intervention therapy for diabetes.

### CROSS REFERENCE TO RELATED APPLICATIONS

### Background of the Invention

(1) Field of the Invention

The present invention relates to the use of N-linked glycosylation profiles of serum proteins as a biomarker for evaluating the efficacy of intervention therapies for diabetes. In particular, the present invention relates to measuring changes in the N-linked glycosylation of total serum plasma proteins over time following the start of an intervention therapy for diabetes to evaluate the efficacy of the intervention therapy for diabetes.

(2) Description of Related Art

Glycated (glycosylated) hemoglobins have gained acceptance as a relevant index of long-term blood glucose control in patients with diabetes mellitus. As used subsequently in this specification the term glycated hemoglobin refers to relatively stable condensation products of hemoglobin with glucose (and possibly glucose phosphates), as compared with more labile hemoglobin-glucose adducts, supposedly of the aldimine (Schiff base) type and generated by a non-enzymatic reaction between glucose and amino groups of hemoglobin. The latter are believed to be converted into the stable (formerly termed “glycosylated”) type via an Amadori rearrangement (cf. M. Roth: Clin. Chem. 29 (1983) 1991).

Glycated hemoglobin A components were first recognized when hemoglobin A was subjected to electrophoresis and cation exchange chromatography. Owing to their more negative charge and consequently higher electrophoretic migration rates towards the anode than that of the major component hemoglobin A (HbAo) they were named the “fast” hemoglobins (HbA1). The fast hemoglobins constitute a series of minor hemoglobins among which inter alia HbA1a, HbA1b and HbA1c have been identified according to their differential migration rates. Of these HbA1c is present in greatest quantity in erythrocytes both from normal subjects and from diabetic patients. HbA1c is known to be glycated at the N-terminal valine of the beta-chains of hemoglobin A. However, recent studies have indicated that glycation may also occur at the amino group of lysine side chains and that all hemoglobins, including HbAo and HbA1c, may comprise such glycated sites. The labile (aldimine) precursor of HbA1c (usually referred to as “pre-HbA1c”) is not encompassed by the above definition of HbA1c.

It is now generally accepted that the level of HbA1c in a blood sample is a good index for the individual's glycemic control. Normal adults have about 90 percent of their total hemoglobin A as HbAo and 3-6 percent as HbA1c, the balance consisting of other minor hemoglobins including HbA1a and HbA1b. However, the level of HbA1c in patients with type 1 (juvenile) and type 2 (maturity-onset) diabetes ranges from about 6 percent to about 15 percent.

The quantification of the HbA1c level in diabetic patients is regarded as a useful means of assessing the adequacy of diabetes control, in that such measurements represent time-averaged values for blood glucose over the preceding 2-4 months (cf. J. S. Schwartz et al.: Annals of Intern. Med. 101 (1984) 710-713). However, changes in HbA1c levels are somewhat delayed in response to the start of an anti-diabetic therapy or treatment, therefore, there remains a desire for identifying other molecules that might precede or predict the subsequent changes in HbA1c.

### BRIEF SUMMARY OF THE INVENTION

The present invention provides for use of the N-linked glycosylation composition of serum proteins as a biomarker for evaluating the efficacy of intervention therapies for diabetes. In particular, the present invention provides that the determining of the changes in the N-glycan composition of total serum plasma proteins over time following the start of an intervention therapy for diabetes may be used to evaluate the efficacy of the intervention therapy.

The inventors have discovered that the N-linked glycosylation pattern or composition of total plasma proteins (or total N-glycan composition) of a plasma or serum sample obtained from a diabetic individual or patient will change over time in response to an intervention therapy for diabetes that is efficacious. The change in N-linked glycosylation pattern or composition of total serum protein (or total N-glycan composition) precedes the decrease in glycated hemoglobin (HbA1c) associated with successful resolution of diabetes by up to 3 weeks in mice. Thus, monitoring or measuring the change in the N-linked glycosylation pattern or composition of total serum proteins (or total N-glycan composition) in serum or plasma samples obtained from a diabetic individual or patient undergoing a diabetes intervention therapy over time may be used to predict the decrease in HbA1c associated with successful resolution of diabetes. Furthermore, determining the change in N-linked glycosylation pattern or composition of total serum proteins (or total N-glycan composition) following the start of an anti-diabetic intervention therapy may be used to evaluate the efficacy of the intervention therapy independent of determining the change in HbA1c.

Therefore, the present invention provides a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition of the serum sample to the N-glycan composition of a serum sample obtained from the individual or patient at a time before the start of the anti-diabetic therapy or treatment, wherein a difference between the N-glycan composition of the serum sample obtained from the individual or patient at a time following the start of the anti-diabetic therapy or treatment and the N-glycan composition of the serum sample obtained from the individual or patient at a time before the start of the anti-diabetic therapy or treatment indicates that the anti-diabetic therapy or treatment is efficacious. In certain embodiments, the difference in N-glycan composition may be detected as a quantitative increase or decrease in the amount of one or more N-glycans or as a trend of increasing or decreasing amount of one or more N-glycans, regardless of the statistical significance of the difference. Alternatively, the difference in N-glycan composition may be detected as a statistically significant increase or decrease in amount of one or more N-glycans.

In a further aspect, the present invention provides a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one high mannose N-glycan, hybrid N-glycan, complex N-glycan, or O-acetylated N-glycan in the N-glycan composition of the serum sample obtained from the individual or patient at a time following the start of the anti-diabetic therapy or treatment compared to the amount of the corresponding high mannose N-glycan, hybrid N-glycan, complex N-glycan, or O-acetylated N-glycan in the N-glycan composition of the serum sample obtained from the individual or patient at a time before the start of the anti-diabetic therapy or treatment indicates that the anti-diabetic therapy or treatment is efficacious. In certain embodiments, such differences may be detected as a quantitative decrease in the amount of at least one high mannose N-glycan, hybrid N-glycan, complex N-glycan or O-acetylated N-glycan, or as a trend of decreasing amount of these N-glycans, regardless of the statistical significance of the decrease. Alternatively, the differences may be detected as a statistically significant decrease in the amount of at least one high mannose N-glycan, hybrid N-glycan, complex N-glycan or O-acetylated N-glycan.

In a further aspect, the present invention provides a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time before the start of the anti-diabetic therapy or treatment, wherein a decrease in the amount of at least one high mannose N-glycan in the N-glycan composition of the serum sample obtained from the individual or patient at a time following the start of the anti-diabetic therapy or treatment compared to the amount of the corresponding high mannose N-glycan in the N-glycan composition of the serum sample obtained from the individual or patient at a time before the start of the anti-diabetic therapy or treatment indicates that the anti-diabetic therapy or treatment is efficacious. In certain embodiments, the decrease in amount may be detected as a quantitative decrease in the amount of at least one high mannose N-glycan, or as a trend of decreasing amount of at least one high mannose N-glycan, regardless of the statistical significance of the decrease. Alternatively, the decrease may be detected as a statistically significant decrease in the amount of at least one high mannose N-glycan.

In particular embodiments of the above, the high mannose N-glycans are selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

In a further aspect, the present invention provides a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one hybrid N-glycan in the N-glycan composition of the serum sample obtained from the individual or patient at a time following the start of the anti-diabetic therapy or treatment compared to the amount of the corresponding hybrid N-glycan in the N-glycan composition of the serum sample obtained from the individual or patient at a time before the start of the anti-diabetic therapy or treatment indicates that the anti-diabetic therapy or treatment is efficacious. In certain embodiments, the decrease in amount may be detected as a quantitative decrease in the amount of at least one hybrid N-glycan, or as a trend of decreasing amount of at least one hybrid N-glycan, regardless of the statistical significance of the decrease. Alternatively, the decrease may be detected as a statistically significant decrease in the amount of at least one hybrid N-glycan.

In particular embodiments of the above, the hybrid N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In a further aspect, the present invention provides a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one complex N-glycan in the N-glycan composition of the serum sample obtained from the individual or patient at a time following the start of the anti-diabetic therapy or treatment compared to the amount of the corresponding complex N-glycan in the N-glycan composition of the serum sample obtained from the individual or patient at a time before the start of the anti-diabetic therapy or treatment indicates that the anti-diabetic therapy or treatment is efficacious. In certain embodiments, the decrease in amount may be detected as a quantitative decrease in the amount of at least one complex N-glycan, or as a trend of decreasing amount of at least one complex N-glycan, regardless of the statistical significance of the decrease. Alternatively, the decrease may be detected as a statistically significant decrease in the amount of at least one complex N-glycan.

In particular embodiments of the above, the complex N-glycan is Sia₂Gal₂GlcNAc₂Man₃GlcNAc₂ (540020), wherein Sia is Neu5Ac or Neu5Gc.

In a further aspect, the present invention provides a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one O-acetylated N-glycan in the N-glycan composition of the serum sample obtained from the individual or patient at a time following the start of the anti-diabetic therapy or treatment compared to the amount of the corresponding O-acetylated N-glycan in the N-glycan composition of the serum sample obtained from the individual or patient at a time before the start of the anti-diabetic therapy or treatment indicates that the anti-diabetic therapy or treatment is efficacious. In certain embodiments, the decrease in amount may be detected as a quantitative decrease in the amount of at least one O-acetylated N-glycan, or as a trend of decreasing amount of at least one O-acetylated N-glycan, regardless of the statistical significance of the decrease. Alternatively, the decrease may be detected as a statistically significant decrease in the amount of at least one O-acetylated N-glycan.

In particular embodiments of the above, the O-acetylated (O-Ac) N-glycans are selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In further embodiments of the above, the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight mass spectrometry (MALDI-TOF MS). In a further embodiment, the MALDI-TOF MS provides data that is analyzed by a computer to provide the N-glycan composition.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a difference between the first and second N-linked glycosylation profiles indicates that the anti-diabetic therapy or treatment is efficacious. An N-linked glycosylation profile is the N-linked glycosylation pattern or signature for the serum sample and comprises a quantitation of the relative amounts of the N-glycans detected in the serum sample. In certain embodiments, the difference between the first and second N-linked glycosylation profiles may be a quantitative increase or decrease in the amount of one or more N-glycans or a trend of increasing or decreasing amount of one or more N-glycans, regardless of the statistical significance of the difference. Alternatively, the difference in N-linked glycosylation profile may be a statistically significant increase or decrease in amount of one or more N-glycans.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a decrease in the amount of at least one high mannose N-glycan, hybrid N-glycan, complex N-glycan, or O-acetylated N-glycan in the second N-linked glycosylation profile compared to the amount of the corresponding high mannose N-glycan, hybrid N-glycan, complex N-glycan, or O-acetylated N-glycan in the first N-linked glycosylation profile indicates that the anti-diabetic therapy or treatment is efficacious. In certain embodiments, the difference between the first and second N-linked glycosylation profiles may be a quantitative decrease in the amount of at least one high mannose N-glycan, hybrid N-glycan, complex N-glycan, or O-acetylated N-glycan, or a trend of decreasing amount of at least one high mannose N-glycan, hybrid N-glycan, complex N-glycan, or O-acetylated N-glycan, regardless of the statistical significance of the decrease. Alternatively, the difference between the first and second N-linked glycosylation profiles may be a statistically significant decrease in amount of at least one mannose N-glycan, hybrid N-glycan, complex N-glycan, or O-acetylated N-glycan.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a decrease in the amount of at least one high mannose N-glycan in the second N-linked glycosylation profile compared to the amount of the corresponding high mannose N-glycan in the first N-linked glycosylation profile indicates that the anti-diabetic therapy or treatment is efficacious. In certain embodiments, the difference between the first and second N-linked glycosylation profiles may be a quantitative decrease in the amount of at least one high mannose N-glycan or a trend of decreasing amount of at least one high mannose N-glycan, regardless of the statistical significance of the decrease. Alternatively, the difference between the first and second N-linked glycosylation profiles may be a statistically significant decrease in amount of at least one high mannose N-glycan.

In particular embodiments of the above, the high mannose N-glycans are selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a decrease in the amount of at least one hybrid N-glycan in the second N-linked glycosylation profile compared to the amount of the corresponding hybrid N-glycan in the first N-linked glycosylation profile indicates that the anti-diabetic therapy or treatment is efficacious. In certain embodiments, the difference between the first and second N-linked glycosylation profiles may be a quantitative decrease in the amount of at least one hybrid N-glycan or a trend of decreasing amount of at least one hybrid N-glycan, regardless of the statistical significance of the decrease. Alternatively, the difference between the first and second N-linked glycosylation profiles may be a statistically significant decrease in amount of at least one hybrid N-glycan.

In particular embodiments of the above, the hybrid N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a decrease in the amount of at least one complex N-glycan in the second N-linked glycosylation profile compared to the amount of the corresponding complex N-glycan in the first N-linked glycosylation profile indicates that the anti-diabetic therapy or treatment is efficacious. In certain embodiments, the difference between the first and second N-linked glycosylation profiles may be a quantitative decrease in the amount of at least one complex N-glycan or a trend of decreasing amount of at least one complex N-glycan, regardless of the statistical significance of the decrease. Alternatively, the difference between the first and second N-linked glycosylation profiles may be a statistically significant decrease in amount of at least one complex N-glycan.

In particular embodiments of the above, the complex N-glycan is Sia₂Gal₂GlcNAc₂Man₃GlcNAc₂ (540020), wherein Sia is Neu5Ac or Neu5Gc.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a decrease in the amount of at least one O-acetylated N-glycan in the second N-linked glycosylation profile compared to the amount of the corresponding O-acetylated N-glycan in the first N-linked glycosylation profile indicates that the anti-diabetic therapy or treatment is efficacious. In certain embodiments, the difference between the first and second N-linked glycosylation profiles may be a quantitative decrease in the amount of at least one O-acetylated N-glycan or a trend of decreasing amount of at least one O-acetylated N-glycan, regardless of the statistical significance of the decrease. Alternatively, the difference between the first and second N-linked glycosylation profiles may be a statistically significant decrease in amount of at least one O-acetylated N-glycan.

In particular embodiments of the above, the O-acetylated (O-Ac) N-glycans are selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In further embodiments of the above, the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight (MALDI-TOF) to provide the N-linked glycosylation profile. In a further embodiment, the MALDI-TOF provides data that is analyzed by a computer to provide the N-linked glycosylation profile.

In a further embodiments of the above, the N-glycan composition obtained from the individual or patient at a time following the start of the therapy or treatment comprises an increase in the amount of one or more fucosylated N-glycans compared to amount of the corresponding fucosylated N-glycan in a serum sample obtained from the individual or patient before the start of the therapy or treatment. In particular aspects, the fucosylated N-glycans are selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc. In certain embodiments, the increase in amount may be detected as a quantitative increase in the amount of at least one fucosylated N-glycan, or as a trend of increasing amount of at least one fucosylated N-glycan, regardless of the statistical significance of the increase. Alternatively, the increase may be detected as a statistically significant increase in the amount of at least one fucosylated N-glycan.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the antidiabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (iv) a decrease in a complex N-glycan such as Sia₂Gal₂GlcNAc₂Man₃GlcNAc₂ (540020), wherein Sia is Neu5Ac or Neu5Gc, indicates that the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (iii) a decrease in a complex N-glycan such as Sia₂Gal₂GlcNAc₂Man₃GlcNAc₂ (540020), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (ii) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000) indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (ii) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (ii) a decrease in a complex N-glycan such as Sia₂Gal₂GlcNAc₂Man₃GlcNAc₂ (540020), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and

Man₅GlcNAc₂ (520000); and (iii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and

Man₅GlcNAc₂ (520000); (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (iv) a decrease in a complex N-glycan such as Sia₂Gal₂GlcNAc₂Man₃GlcNAc₂ (540020), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Man₉GlcNAc₂ (920000), MangGlcNAc₂ (820000), Man₇GlcNAc₂ (720000), Man₆GlcNAc₂ (620000), and Man₅GlcNAc₂ (520000); (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (iv) a decrease in a complex N-glycan such as Sia₂Gal₂GlcNAc₂Man₃GlcNAc₂ (540020), wherein Sia is Neu5Ac or Neu5Gc; and (v) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Man₉GlcNAc₂ (920000), MangGlcNAc₂ (820000), Man₇GlcNAc₂ (720000), Man₆GlcNAc₂ (620000), and Man₅GlcNAc₂ (520000); (iii) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (iv) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (iii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (iii) a decrease in a complex N-glycan such as Sia₂Gal₂GlcNAc₂Man₃GlcNAc₂ (540020), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (ii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (iii) a decrease in a complex N-glycan such as Sia₂Gal₂GlcNAc₂Man₃GlcNAc₂ (540020), wherein Sia is Neu5Ac or Neu5Gc; and (iv) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (ii) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000) indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (ii) a decrease in a complex N-glycan such as Sia₂Gal₂GlcNAc₂Man₃GlcNAc₂ (540020), wherein Sia is Neu5Ac or Neu5Gc; and (iii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (ii) a decrease in a complex N-glycan such as Sia₂Gal₂GlcNAc₂Man₃GlcNAc₂ (540020), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in a complex N-glycan such as Sia₂Gal₂GlcNAc₂Man₃GlcNAc₂ (540020), wherein Sia is Neu5Ac or Neu5Gc; and (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc, indicates the anti-diabetic therapy or treatment is efficacious.

In a further aspect, the present invention provides a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (c) comparing the first and second N-linked glycosylation profiles, wherein (i) a decrease in a complex N-glycan such as Sia₂Gal₂GlcNAc₂Man₃GlcNAc₂ (540020), wherein Sia is Neu5Ac or Neu5Gc; and (ii) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000) indicates the anti-diabetic therapy or treatment is efficacious.

In further embodiments of any one of the above aspects or embodiments, one or more serum samples are or were obtained from the individual or patient from a time selected from 3, 4, 5, 6, 7, 8, 9, 10, 11, 12, 13, 14, 15, 16, 17, 18, 19, 20, and 21 days following the start of the therapy or treatment. In particular aspects, serum samples were obtained from the individual or patient from a time selected from about day 7 and/or about day 14 following the start of the therapy or treatment.

In particular embodiments of the above, the anti-diabetic therapy or treatment comprises an insulin, an insulin sensitizer, insulin secretagogue, alpha-glucosidase inhibitor, incretin or incretin mimetic, dipeptidyl peptidase 4 (DPP4) inhibitor, amylin or amylin analog, or GLP-1 receptor agonist. Insulin sensitizers include but are not limited to biguanides and thiazolidinediones wherein the biguanides include but are not limited to metformin, phenformin, and buformin and the thiazolidinediones include but are not limited to rosiglitazone, pioglitazone, and troglitazone. The insulin secretagogues include but are not limited to sulfonylureas and non-sulfonylureas wherein the sulfonylureas include but are not limited to tolbutamide, acetohexamide, tolazamide, chlorpropamide, glipizide, glyburide, glimepiride, and gliclazide and the non-sulfonylurease include but are not limited to metglitinides such as repaglinide and nateglinide. Alpha-glucosidase inhibitors include but are not limited to miglitol and acarbose. Incretin or incretin mimetics include but are not limited to GLP1 receptor agonists such as GLP1, oxyntomodulin, exenatide, liraglutide, taspoglutide, and glucagon analogs that have GLP1 receptor agonist activity. DPP4 inhibitors include but are not limited to vildagliptin, sitagliptin, saxagliptin, and linagliptin.

Thus, in light of the above, the present invention provides a biomarker for determining efficacy of a treatment for diabetes which comprises the N-linked glycosylation profile of the proteins in plasma or serum.

The present invention further provides for the use of an N-linked glycosylation profile of a serum sample from an individual or patient in which an anti-diabetic therapy or treatment has been initiated as a predictive biomarker for determining efficacy of the therapy or treatment for diabetes.

The present invention further provides for the use of the amount of one or more high mannose and/or hybrid N-glycans in a serum sample obtained from an individual or patient in which an anti-diabetic therapy or treatment has been initiated as a predictive biomarker for determining efficacy of the therapy or treatment for diabetes.

The present invention further provides for the use of the amount of one or more high mannose N-glycans, hybrid N-glycans, O-acetylated N-glycans, complex N-glycans, fucosylated N-glycans, or combinations thereof in a serum sample obtained from an individual or patient in which an anti-diabetic therapy or treatment has been initiated as a predictive biomarker for determining efficacy of the therapy or treatment for diabetes.

### DEFINITIONS

As used herein, the terms “N-glycan” and “N-linked glycan” are used interchangeably and refer to an N-glycan in which the N-acetylglucosamine residue at the reducing end that may be linked in a 131 linkage to the amide nitrogen of an asparagine residue of an attachment group in the protein. Thus, the term refers to the N-glycan whether it is attached to the protein or has been detached from the protein.

As used herein, the terms “N-linked glycosylated” and “N-glycosylated” are used interchangeably and refer to an N-glycan attached to an attachment group comprising an asparagine residue or an N-linked glycosylation site or motif

As used herein, “N-glycans” are oligosaccharides that have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that may be attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid (“Sia”) or derivatives (e.g., “NANA” or “NeuAc” where “Neu” refers to neuraminic acid and “Ac” refers to acetyl, or the derivative NGNA, which refers to N-glycolylneuraminic acid). Complex N-glycans may also have intrachain substitutions comprising “bisecting” GlcNAc and core fucose (“Fuc”). Complex N-glycans may also have multiple antennae on the “trimannose core,” often referred to as “multiple antennary N-glycans.” A “hybrid” N-glycan has at least one GlcNAc on the terminal of the 1,3 mannose arm of the trimannose core, no GlcNAc on the 1,6 mannose arm, and zero or more mannoses on the 1,6 mannose arm of the trimannose core. FIG. 2 shows the symbols and nomenclature used to represent the various sugars comprising N-glycan structures.

The term “fucosylated glycan” or “fucosylated N-glycan” refers to any N-glycan that has one or more fucose residue(s) anywhere on the structure, including, but not limited to core fucose. The term “O-acetylated glycan” or “O-acetylated N-glycan” refers to any N-glycan that has one of the hydroxyl groups esterified with an acetyl group or more than one hydroxyl group, each esterified with an acetyl group.

N-glycans consisting of a Man₃GlcNAc₂ structure are called paucimannose. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as SiaGal₂GlcNAc₂Man₃GlcNAc₂; and, the term “A2” refers to an N-glycan structure that can be characterized as Sia₂Gal₂GlcNAc₂Man₃GlcNAc₂. Unless otherwise indicated, the terms G-2″, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contain a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that contain fucose.

As used herein, the structure of an N-glycan may be expressed using a six-digit identifier. The six-digit identifiers are interpreted as follows: the first digit indicates the number of hexoses in the structure (i.e., mannose, galactose or glucose); the second digit indicates the number of N-acetylhexosamines in the structure (i.e., GlcNAc or GalNAc); the third digit indicates the number of deoxyhexoses in the structure (i.e., fucose); the fourth digit indicates the number of N-acetylneuraminic acids (Neu5Ac) in the structure; the fifth digit indicates the number of N-glycolylneuraminic acids (Neu5Gc) in the structure, and; the sixth digit indicates the number of O-acetates (OAc) in the structure. Alternatively, the structure of an N-glycan may be illustrated using the nomenclature developed by the Consortium of Functional Glycomics, as is known in the art and illustrated in FIG. 2.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or Sia₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the non-reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

Abbreviations used herein are of common usage in the art, see, e.g., abbreviations of sugars, above. Other common abbreviations include “PNGase”, or “glycanase” which all refer to glycopeptide N-glycosidase; glycopeptidase; N-oligosaccharide glycopeptidase; N-glycanase; glycopeptidase; Jack-bean glycopeptidase; PNGase A; PNGase F; glycopeptide N-glycosidase (EC 3.5.1.52, formerly EC 3.2.2.18).

As used herein, the term “insulin” means the active principle of the pancreas that affects the metabolism of carbohydrates in the animal body and which is of value in the treatment of diabetes mellitus. The term includes synthetic and biotechnologically-derived products that are the same as, or similar to, naturally occurring insulins in structure, use, and intended effect and are of value in the treatment of diabetes mellitus.

The term “insulin” or “insulin molecule” is a generic term that designates the 51 amino acid heterodimer comprising an A-chain peptide and a B-chain peptide.

The term “insulin analogue” as used herein includes any heterodimer analogue or single-chain analogue that comprises one or more modification(s) of the native A-chain peptide and/or B-chain peptide. Modifications include but are not limited to any amino acid substitution or deletion at any position in the A-chain peptide, B-chain peptide, and/or C-peptide or conjugating directly or by a polymeric or non-polymeric linker one or more acyl, polyethylglycine (PEG), or saccharide moiety (moieties); or any combination thereof. The term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473 and 6,630,348; and Kristensen et al., Biochem. J. 305: 981-986 (1995), the disclosures of which are each incorporated herein by reference.

### BRIEF DESCRIPTION OF THE DRAWINGS

FIG. 1 shows a schematic representation of a protocol that may be used to determine the N-glycan composition or pattern of total proteins in a complex biological sample.

FIG. 2 shows the symbols and nomenclature used to represent the various sugars comprising N-glycan structures.

FIGS. 3A-3E show that various high mannose N-glycans were lower in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 3A shows that Glycan 520000 is lower in rosiglitazone-treated db/db mice compared to vehicle-treated db/db mice. FIG. 3B shows that Glycan 620000 is lower in rosiglitazone-treated db/db mice compared to vehicle-treated db/db mice. FIG. 3C shows that Glycan 720000 is lower in rosiglitazone-treated db/db mice compared to vehicle-treated db/db mice. FIG. 3D shows that Glycan 820000 is lower in rosiglitazone-treated db/db mice compared to vehicle-treated db/db mice. FIG. 3E shows that Glycan 920000 is lower in rosiglitazone-treated db/db mice compared to vehicle-treated db/db mice.

FIGS. 4A-4C show that various fucosylated N-glycans were higher in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 4A show that Glycan 651030 exhibits a significant increase in rosiglitazone-treated db/db mice compared to vehicle-treated db/db mice. FIG. 4B shows that Glycan 651031 exhibits a significant increase in rosiglitazone-treated db/db mice compared to vehicle-treated db/db mice. FIG. 4C shows that Glycan 761040 exhibits a significant increase in rosiglitazone-treated db/db mice compared to vehicle-treated db/db mice. Glycan 761040 was below the limit of quantitation (LOQ) in some samples, preventing statistical analysis at some time points.

FIGS. 5A-5D show that various O-acetylated N-glycans were lower in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 5A shows that Glycan 540021 is lower in rosiglitazone-treated db/db mice compared to vehicle-treated db/db mice. FIG. 5B shows that Glycan 540022 is lower in rosiglitazone-treated db/db mice compared to vehicle-treated db/db mice. FIG. 5C shows that Glycan 540031 is lower in rosiglitazone-treated db/db mice compared to vehicle-treated db/db mice. FIG. 5D shows that Glycan 540032 is lower in rosiglitazone-treated db/db mice compared to vehicle-treated db/db mice.

FIGS. 6A-6C show that various hybrid N-glycans were lower in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 6A shows that Glycan 430010 is lower in rosiglitazone-treated db/db mice compared to vehicle-treated db/db mice. FIG. 6B shows that Glycan 530010 is lower in rosiglitazone-treated db/db mice compared to vehicle-treated db/db mice. FIG. 6C shows that Glycan 630010 is lower in rosiglitazone-treated db/db mice compared to vehicle-treated db/db mice.

FIG. 7 shows that Glycan 540020 is lower in rosiglitazone-treated db/db mice compared to vehicle-treated db/db mice. The graph plots the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001.

FIGS. 8A-8E are scatter plots showing that various high mannose N-glycans were lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 8A shows that Glycan 520000 is lower in rosiglitazone-treated db/db mice compared to vehicle-treated db/db mice. FIG. 8B shows that Glycan 620000 is lower in rosiglitazone-treated db/db mice compared to vehicle-treated db/db mice. FIG. 8C shows that Glycan 720000 is lower in rosiglitazone-treated db/db mice compared to vehicle-treated db/db mice. FIG. 8D shows that Glycan 820000 is lower in rosiglitazone-treated db/db mice compared to vehicle-treated db/db mice. FIG. 8E shows that Glycan 920000 is lower in rosiglitazone-treated db/db mice compared to vehicle-treated db/db mice.

FIGS. 9A-9C are scatter plots showing that various fucosylated N-glycans were higher in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 9A show that Glycan 651030 exhibits a significant increase in rosiglitazone-treated db/db mice compared to vehicle-treated db/db mice. FIG. 9B shows that Glycan 761040 exhibits a significant increase in rosiglitazone-treated db/db mice compared to vehicle-treated db/db mice. FIG. 9C shows that Glycan 651031 exhibits a significant increase in rosiglitazone-treated db/db mice compared to vehicle-treated db/db mice.

FIGS. 10A-10D are scatter plots showing that various O-acetylated N-glycans were lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 10A shows that Glycan 540021 is lower in rosiglitazone-treated db/db mice compared to vehicle-treated db/db mice. FIG. 10B shows that Glycan 540022 is lower in rosiglitazone-treated db/db mice compared to vehicle-treated db/db mice. FIG. 10C shows that Glycan 540031 is lower in rosiglitazone-treated db/db mice compared to vehicle-treated db/db mice. FIG. 10D shows that Glycan 540032 is lower in rosiglitazone-treated db/db mice compared to vehicle-treated db/db mice.

FIGS. 11A-11C are scatter plots showing that various hybrid N-glycans were lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 11A shows that Glycan 430010 is lower in rosiglitazone-treated db/db mice compared to vehicle-treated db/db mice. FIG. 11B shows that Glycan 530010 is lower in rosiglitazone-treated db/db mice compared to vehicle-treated db/db mice. FIG. 11C shows that Glycan 630010 is lower in rosiglitazone-treated db/db mice compared to vehicle-treated db/db mice.

FIG. 12 is a scatter plot showing that Glycan 540020 is lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001.

FIGS. 13A-13D show that various high mannose N-glycans were lower in insulin detemir-treated db/db mice compared to vehicle-treated db/db mice. The graphs plot the mean of all samples over time, with error bars representing the standard error. Statistical significance of the difference between insulin detemir-treated and vehicle-treated db/db mice at each time point is indicated by asterisks, where *=p<0.05, **=p<0.01, and ***=p<0.001. FIG. 13A shows that Glycan 520000 is lower in insulin detemir-treated db/db mice compared to vehicle-treated db/db mice. FIG. 13B shows that Glycan 620000 is lower in insulin detemir-treated db/db mice compared to vehicle-treated db/db mice. FIG. 13C shows that Glycan 720000 is lower in insulin detemir-treated db/db mice compared to vehicle-treated db/db mice. FIG. 13D shows that Glycan 820000 is lower in insulin detemir-treated db/db mice compared to vehicle-treated db/db mice.

FIGS. 14A-14C show that various hybrid N-glycans were lower in insulin detemir-treated db/db mice compared to vehicle-treated db/db mice. The graphs plot the mean of all samples over time, with error bars representing the standard error. Statistical significance of the difference between insulin detemir-treated and vehicle-treated db/db mice at each time point is indicated by asterisks, where *=p<0.05, **=p<0.01, and ***=p<0.001. FIG. 14A shows that Glycan 430010 is lower in insulin detemir-treated db/db mice compared to vehicle-treated db/db mice. FIG. 14B shows that Glycan 530010 is lower in insulin detemir-treated db/db mice compared to vehicle-treated db/db mice. FIG. 14C shows that Glycan 630010 is lower in insulin detemir-treated db/db mice compared to vehicle-treated db/db mice.

### DETAILED DESCRIPTION OF THE INVENTION

The present invention provides a biomarker for determining the efficacy of an anti-diabetic therapy or treatment regime. The biomarker comprises the N-linked glycosylation composition of total serum proteins in a serum sample obtained from an individual or patient undergoing an anti-diabetic therapy or treatment regime wherein the amount of one or more particular N-glycans in the composition increase or decrease over time compared to the N-linked glycosylation composition of total serum proteins in a serum sample obtained from the individual or patient prior to the start of the anti-diabetic therapy or treatment regime. In general, the increase and/or decrease in the amounts of particular N-glycans in the composition occurs between 3 and 14 days after the start of the therapy or treatment regime in the db/db mouse model (a generally accepted model for evaluating anti-diabetic treatments), with the increase and/or decrease of the N-glycan amounts in the composition stabilizing by about day 14 after the start of the therapy or treatment regime. Thus, the present invention provides a biomarker for evaluating glycemic control of an anti-diabetic therapy or treatment regime.

While glycemic control is routinely evaluated by monitoring changes in HbA1c levels over time in patients undergoing a therapy or treatment regime for diabetes, in general the changes in HbA1c levels are delayed relative to the start of the therapy or treatment. Therefore, it is not possible until some time after the commencement of the therapy or treatment to know whether the therapy or treatment chosen is or will be efficacious. To improve therapy or treatment outcomes, it would be desirable to know at an earlier time period following start of the therapy or treatment whether the particular therapy or treatment chosen was efficacious, allowing a non-efficacious therapy or treatment to be modified or replaced with another therapy or treatment at an earlier time period than is currently possible. As disclosed herein, the inventors have discovered that the N-linked glycan pattern, profile, or signature of total serum proteins may be used as a biomarker of changes in HbA1c amounts in serum at an earlier time period in the therapy or treatment. Thus, the present invention provides a biomarker that enables the efficacy of a therapy or treatment regime to be determined at a time period preceding the change in HbA1c amounts in serum.

An initial study (Study 1) was an analysis of 160 plasma samples collected from rosiglitazone-treated mice following oral administration of 10 mpk once daily for up to 39 days. Rosigilitazone is an athiazolidinedione class of antidiabetic drug marketed by Glaxo under the trade name AVANDIA. Rosiglitazone works as an insulin sensitizer, by binding to the peroxisome proliferator-activated receptors (PPAR) receptors in fat cells and making the cells more responsive to insulin) and control (vehicle-treated) mice over a 39 day time course showed that rosiglitazone induced early, dramatic, and sustained changes in the N-glycan profile of plasma proteins. These changes, on an individual N-glycan basis, reached high statistical significance (P<0.001) as early as seven days, a full two weeks prior to changes in HbA1c (day seven vs. day 21, respectively). The drug-induced N-glycan changes in the N-linked glycosylation profiles were found to correlate with the level of HbA1c, and further, these changes clustered in structurally-related groups of N-glycans, suggesting a biosynthetically-linked drug effect. The drug-induced N-glycan changes in N-linked glycosylation profiles can be grouped into three structurally-related categories: Fucosylated N-glycans, which were higher in rosiglitazone-treated db/db mice compared to vehicle controls; high-mannose N-glycans, which were lower in rosiglitazone-treated db/db mice compared to vehicle controls; hybrid glycans, which were lower in rosiglitazone-treated db/db mice compared to vehicle controls; and, O-Acetylated N-glycans, which were lower in rosiglitazone-treated db/db mice compared to vehicle controls. Reduction in high-mannose N-glycans was about a 2-3 week earlier leading indicator of the eventual reduced HbA1c amounts expected in an efficacious therapy or treatment regime, and fucosylated N-glycans were an about one week earlier indicator of the eventual reduced HbA1c amounts expected in an efficacious therapy or treatment regime.

In a second study (Study 2), a total of 48 mouse plasma samples from mice treated daily with vehicle or with 10 mpk rosiglitazone were analyzed at 0 and seven days. The samples analyzed included a day 0 time point, which established an important baseline given the early nature of the changes observed in Study 1. To verify changes observed in Study 1 in a new in vivo experiment, Day 7 samples from an independent set of db/db mice treated with vehicle and rosiglitazone (included in Study 1) were also analyzed. The results showed that the amounts in four high mannose N-glycans, three hybrid N-glycans, four O-acetylated N-glycans, and one complex N-glycan decreased and four fucosylated N-glycans increased in the serum samples obtained from the rosiglitazone-treated db/db mice compared to non-treated controls. The high mannose N-glycans that were decreased in the rosiglitazone-treated db/db mice were Man₉GlcNAc₂ (920000), Man₈GlcNAc₂ (820000), Man₇GlcNAc₂ (720000), Man₆GlcNAc₂ (620000), and Man₅GlcNAc₂ (520000). The hybrid N-glycans that were decreased in the rosiglitazone-treated db/db mice were SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc. The O-acetylated N-glycans that were decreased in the rosiglitazone-treated db/db mice were Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc. The complex N-glycan that was decreased in the rosiglitazone-treated db/db mice was Sia₂Gal₂GlcNAc₂Man₃GlcNAc₂ (540020). The fucosylated N-glycans that were increased in the rosiglitazone-treated db/db mice were Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc.

In Study 3, changes in the N-linked glycosylation profile of total serum proteins obtained from diabetic mice were further characterized using a different drug-treatment to determine the drug specificity of the rosiglitazone-induced changes. In an animal study, insulin detemir was administered subcutaneously in two doses of 10 U/kg per day. Samples were collected from insulin detemir and vehicle-treated db/db mice at four time points (0, 7, 14, and 21 days). (Insulin detemir is a long-acting insulin marketed by Novo Nordisk under the trade name LEVEMIR.). The results shown in Example 3 showed that the amounts of four high mannose N-glycans and three hybrid N-glycans decreased in the serum samples obtained from the insulin-treated db/db mice compared to non-treated controls. No change was observed in fucosylated, O-acetylated, or tetraantennary N-glycans in the serum samples from the insulin-treated db/db mice compared to non-treated controls. The high mannose N-glycans that were decreased in the insulin-treated db/db mice were Man₈GlcNAc₂ (820000), Man₇GlcNAc₂ (720000), Man₆GlcNAc₂ (620000), and Man₅GlcNAc₂ (520000). The hybrid N-glycans that were decreased in the insulin-treated db/db mice were SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

These results demonstrate that the change in the N-linked glycosylation pattern or N-glycan profile of total serum proteins over time in an individual or patient undergoing an anti-diabetic therapy or treatment can be used as a biomarker for evaluating the efficacy of the anti-diabetic therapy or treatment. In general, at least one serum sample is obtained from an individual or patient undergoing an anti-diabetic therapy or treatment at a time following the start of the therapy or treatment. The serum sample is treated with an enzyme such as PNGase F to release the N-glycans from the serum proteins. The N-glycans are then separated from the serum proteins to provide a composition of the N-glycans, which is then analyzed to determine the N-glycan pattern or profile for the serum sample. In one embodiment, the serum sample may be analyzed by Matrix-Assisted Laser Desorption/Ionization-Time-Of-Flight mass specrometry (MALDI-TOF MS), and the MALDI-TOF MS data may be analyzed by computer using a bioinformatics analysis program and the results of the analysis provided in a report showing the N-glycan pattern or profile for the serum sample. In an alternative embodiment, the serum sample may be analyzed by any means which provides the N-glycan pattern or profile of the sample, for example, HPLC. The N-glycan pattern or profile for the serum sample is compared to the N-glycan pattern or profile of a serum sample obtained from the individual before the start of the anti-diabetic therapy or treatment to provide a baseline or control N-glycan pattern or profile. In further embodiments, one or more serum samples are obtained from the individual or patient from a time selected from 3, 4, 5, 6, 7, 8, 9, 10, 11, 12, 13, 14, 15, 16, 17, 18, 19, 20, and 21 days following the start of the therapy or treatment. In particular aspects, serum samples are obtained from the individual or patient from a time selected from about day 7 and/or about day 14 following the start of the therapy or treatment.

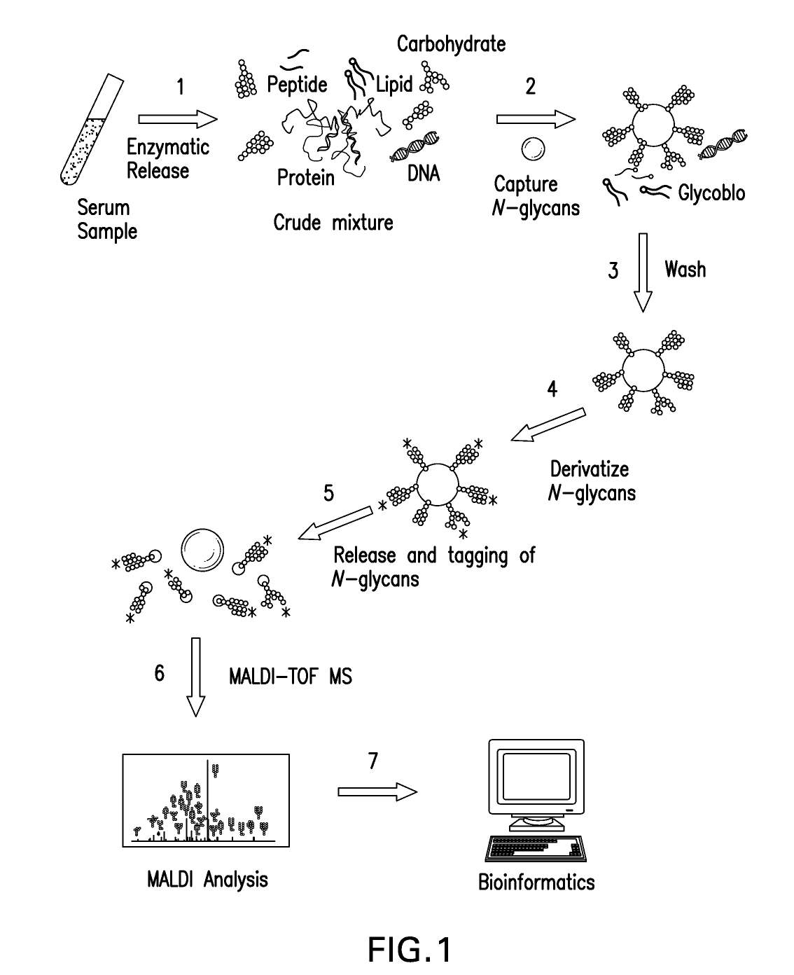

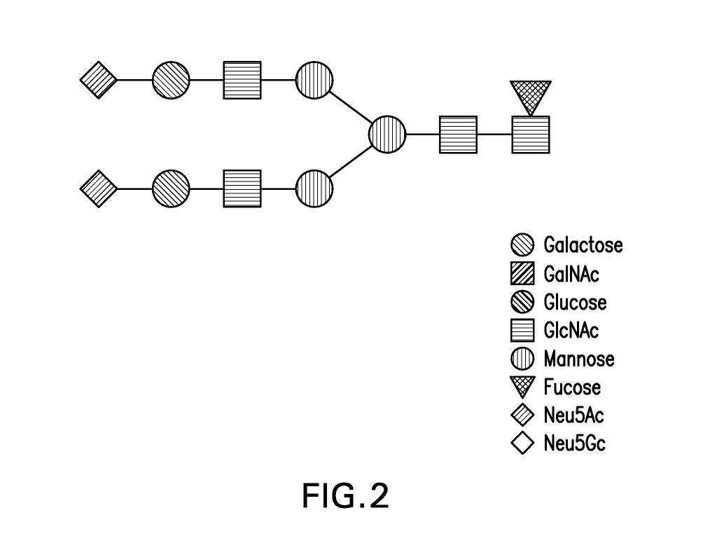

FIG. 1 shows a schematic representation of a protocol that may be used to determine the N-glycan composition or pattern of total proteins in a complex biological sample. Starting from complex biological samples (e.g., serum), each sample is enzymatically treated (Step 1) to provide a crude mixture of released N-glycans, peptides, lipids, and nucleic acids. For example, the samples may be denatured and then digested with trypsin, followed by heat-inactivation, and then digestion with PNGase F (See for example, Papac, et al. Glycobiology 8: 445-454 (1998)). The N-glycans are captured to a solid support that is capable of binding N-glycans and does not bind proteins, polypeptides, peptides, lipids, nucleic acids, or other macromolecules present in the sample (Step 2). In particular embodiments, the solid support are beads (as shown in the figure) comprising aminoxy-functionalized polymers (For example, BLOTGLYCO H beads Sumitomo Bakelite Co., Ltd., Tokyo, Japan) and the N-glycans are bound thereto via oxime bond formation. After thorough washing (Step 3) to remove nonspecifically bound substances, the covalently bound N-glycans are subjected to on-bead methyl esterification to stabilize sialic acids (See for example, Sekiya et al., Anal. Chem. 77: 4962-4968 (2005)) and are recovered in the form of oxime derivatives of the O-substituted aminooxy compound that had been added (Step 4). The N-glycans are simultaneously released from the substrate, labeled (5) and analyzed by MALDI-TOF MS in the positive-ion, reflector mode (Step 6). Methods for performing MALDI-TOF analysis of N-glycans have been disclosed for example in Miele et al. Biotechnol. Appl. Biochem. 25: 151-157 (1997). The results may be analyzed by computer using a bioinformatics program (Step 7). For example, the detected N-glycan peaks in MALDI-TOF-MS spectra may be picked by means of a computer using a software such as FlexAnalysis version 3 (Bruker Daltonics, Billerica, Mass.). Glycan structures may be identified using GlycoMod Tool and GlycoSuite (Tyrian Diagnostics Limited, Sydney, Australia). The above process has been disclosed in the art, for example Nishimura et al. (Angew Chem. Int. Ed. Engl., 44: 91-96 (2004)); Niikura et al. (Chem.-A Eur. J. 11: 3825-3834 (2005); Furukawa et al. (Anal. Chem., 80: 1094-1101 (2008)); Miura et al. (Chem.-A Eur. J, 13: 4797-4804 (2007)); Shimaoka et al. (Chem.-A Eur. J. 13: 1664-1673 (2007)); Miura et al. (Moll. Cell. Proteomics 7: 270-277 (2008)); Amano & Nishimura (Methods Enzymol. 478: 109-125 (2010)); and Aman et al. (ChemBioChem 13: 451-464 (2012)).

In particular embodiments, the change in the N-linked glycosylation pattern or profile of total serum proteins over time in an individual or patient undergoing an anti-diabetic therapy or treatment that is observed comprises a decrease in one or more high mannose N-glycans. In a further aspect, the change in the N-linked glycosylation pattern or profile of total serum proteins over time in an individual or patient undergoing an anti-diabetic therapy or treatment that is observed comprises a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

In particular embodiments, the change in the N-linked glycosylation pattern or profile of total serum proteins over time in an individual or patient undergoing an anti-diabetic therapy or treatment that is observed comprises a decrease in one or more hybrid N-glycans. In a further aspect, the change in the N-linked glycosylation pattern or profile of total serum proteins over time in an individual or patient undergoing an anti-diabetic therapy or treatment that is observed comprises a decrease in one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises a decrease in one or more O-acetylated (O-Ac) N-glycans. In a further aspect, the change in the N-linked glycosylation pattern or profile of total serum proteins over time in an individual or patient undergoing an anti-diabetic therapy or treatment that is observed comprises a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (c) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; (c) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (d) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (c) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises an increase in one or more fucosylated N-glycans. In a further aspect, the change in the N-linked glycosylation pattern or profile of total serum proteins over time in an individual or patient undergoing an anti-diabetic therapy or treatment that is observed comprises an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), MangGlcNAc₂ (820000), Man₇GlcNAc₂ (720000), Man₆GlcNAc₂ (620000), and Man₅GlcNAc₂ (520000); and (c) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (d) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (d) a decrease in a complex N-glycan such as Sia₂Gal₂GlcNAc₂Man₃GlcNAc₂ (540020), wherein Sia is Neu5Ac or Neu5Gc; and (e) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (d) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (c) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (c) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (c) a decrease in a complex N-glycan such as Sia₂Gal₂GlcNAc₂Man₃GlcNAc₂ (540020), wherein Sia is Neu5Ac or Neu5Gc; and (d) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

The use of the change in the N-linked glycosylation pattern or profile of total serum proteins in response to an anti-diabetes therapy or treatment regime as a biomarker for determining the efficacy of the therapy or treatment regime may be suitable for determining the efficacy of any anti-diabetic therapy or treatment regime, including but not limited to anti-diabetic agents such as

(1) PPARγ agonists such as glitazones (e.g. ciglitazone; darglitazone; englitazone; isaglitazone (MCC-555); pioglitazone (ACTOS); rosiglitazone (AVANDIA); troglitazone; rivoglitazone, BRL49653; CLX-0921; 5-BTZD, GW-0207, LG-100641, R483, and LY-300512, and the like and compounds disclosed in WO97/10813, 97/27857, 97/28115, 97/28137, 97/27847, 03/000685, and 03/027112 and SPPARMS (selective PPAR gamma modulators) such as T131 (Amgen), FK614 (Fujisawa), netoglitazone, and metaglidasen; (2) biguanides such as buformin; metformin; and phenformin, and the like; (3) protein tyrosine phosphatase-1B (PTP-1B) inhibitors such as ISIS 113715, A-401674, A-364504, IDD-3, IDD 2846, KP-40046, KR61639, MC52445, MC52453, C7, OC-060062, OC-86839, OC29796, TTP-277BC1, and those agents disclosed in WO 04/041799, 04/050646, 02/26707, 02/26743, 04/092146, 03/048140, 04/089918, 03/002569, 04/065387, 04/127570, and US 2004/167183; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, metiglinide (GLUFAST) and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and AI-3688, and the like; (8) insulin secreatagogues such as linogliride nateglinide, mitiglinide (GLUFAST), ID1101 A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics or analogs, such as biota, LP-100, novarapid, insulin detemir, insulin lispro, insulin aspart, insulin glargine, insulin zinc suspension (lente and ultralente); Lys-Pro insulin, GLP-1 receptor agonists, including but not limited to oxyntomodulin, GLP-1 (17-36), GLP-1 (73-7) (insulintropin); GLP-1 (7-36)-NH2) exenatide/Exendin-4, Exenatide LAR, Linaglutide, AVE0010, CJC 1131, BIM51077, CS 872, TH0318, BAY-694326, GP010, ALBUGON (GLP-1 fused to albumin), HGX-007 (Epac agonist), S-23521, and compounds disclosed in WO 04/022004, WO 04/37859, and the like, and direvatives and analogs thereof; (12) non-thiazolidinediones such as JT-501, and farglitazar (GW-2570/GI-262579), and the like; (13) PPARα/γ dual agonists such as AVE 0847, CLX-0940, GW-1536, GW1929, GW-2433, KRP-297, L-796449, LBM 642, LR-90, LY510919, MK-0767, ONO 5129, SB 219994, TAK-559, TAK-654, 677954 (GlaxoSmithkline), E-3030 (Eisai), LY510929 (Lilly), AK109 (Asahi), DRF2655 (Dr. Reddy), DRF8351 (Dr. Reddy), MC3002 (Maxocore), TY51501 (ToaEiyo), farglitazar, naveglitazar, muraglitazar, peliglitazar, tesaglitazar (GALIDA), reglitazar (JT-501), chiglitazar, and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453, and the like; (14), insulin, insulin mimetics, and other insulin sensitizing drugs; (15) VPAC2 receptor agonists; (16) GLK modulators, such as PSN105, RO 281675, RO 274375 and those disclosed in WO 03/015774, WO 03/000262, WO 03/055482, WO 04/046139, WO 04/045614, WO 04/063179, WO 04/063194, WO 04/050645, and the like; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine, CT21022, CT20026, CT-98023, SB-216763, SB410111, SB-675236, CP-70949, XD4241 and those compounds disclosed in WO 03/037869, 03/03877, 03/037891, 03/024447, 05/000192, 05/019218 and the like; (19) glycogen phosphorylase (HGLPa) inhibitors, such as AVE 5688, PSN 357, GPi-879, those disclosed in WO 03/037864, WO 03/091213, WO 04/092158, WO 05/013975, WO 05/013981, US 2004/0220229, and JP 2004-196702, and the like; (20) ATP consumption promotors such as those disclosed in WO 03/007990; (21) fixed combinations of PPARγ agonists and metformin such as AVANDAMET; (22) PPAR pan agonists such as GSK 677954; (23) GPR40 (G-protein coupled receptor 40) also called SNORF 55 such as BG 700, and those disclosed in WO 04/041266, 04/022551, 03/099793; (24) GPR119 (G-protein coupled receptor 119, also called RUP3; SNORF 25) such as RUP3, HGPRBMY26, PFI 007, SNORF 25; (25) adenosine receptor 2B antagonists such as ATL-618, AT1-802, E3080, and the like; (26) carnitine palmitoyl transferase inhibitors such as ST 1327, and ST 1326, and the like; (27) Fructose 1,6-bisphospohatase inhibitors such as CS-917, MB7803, and the like; (28) glucagon antagonists such as AT77077, BAY 694326, GW 4123X, NN2501, and those disclosed in WO 03/064404, WO 05/00781, US 2004/0209928, US 2004/029943, and the like; (29) glucose-6-phosphase inhibitors; (30) phosphoenolpyruvate carboxykinase (PEPCK) inhibitors; (31) pyruvate dehydrogenase kinase (PDK) activators; (32) RXR agonists such as MC1036, CS00018, JNJ 10166806, and those disclosed in WO 04/089916, U.S. Pat. No. 6,759,546, and the like; (33) SGLT inhibitors such as AVE 2268, KGT 1251, T1095/RWJ 394718; (34) BLX-1002; (35) alpha glucosidase inhibitors; (36) glucagon receptor agonists; (37) glucokinase activators; (38) GIP-1; glucagon/GLP-1 receptor dual agonists, including but not limited to those disclose in WO2008/101017, WO2009/155258, WO2007/100535, WO2011/094337, WO2011/075393, WO2010/096142, WO2010/096052, WO2011/162968, W02011/1432208, WO2011/143209, and (39) insulin secretagogues.

In particular embodiments, provided is a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a difference in the N-glycan composition indicates that the anti-diabetic therapy or treatment is efficacious.

In particular embodiments, provided is a method of determining the efficacy of an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one high mannose N-glycan in the N-glycan composition obtained from the serum sample obtained from the individual or patient at a time following the start of the anti-diabetic therapy or treatment indicates that the anti-diabetic therapy or treatment is efficacious.

In particular embodiments, provided is a method of determining the efficacy of an an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one hybrid N-glycan in the N-glycan composition obtained from the serum sample obtained from the individual or patient at a time following the start of the anti-diabetic therapy or treatment indicates that the anti-diabetic therapy or treatment is efficacious.

In particular embodiments, provided is a method of determining the efficacy of an an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one complex N-glycan in the N-glycan composition obtained from the serum sample obtained from the individual or patient at a time following the start of the anti-diabetic therapy or treatment indicates that the anti-diabetic therapy or treatment is efficacious.

In particular embodiments, provided is a method of determining the efficacy of an an anti-diabetic therapy or treatment comprising (a) determining the N-glycan composition of a serum sample obtained from an individual or patient at a time following the start of the anti-diabetic therapy or treatment; and (b) comparing the N-glycan composition to the N-glycan composition of a serum sample obtained from the individual or patient at a time period before the start of the anti-diabetic therapy or treatment, wherein a decrease in the amount of at least one O-acetylated N-glycan in the N-glycan composition obtained from the serum sample obtained from the individual or patient at a time following the start of the anti-diabetic therapy or treatment indicates that the insulin sensitizer in an anti-diabetic therapy or treatment is efficacious.

In particular embodiments, provided is a method of determining the efficacy of an anti-diabetic therapy or treatment comprising (a) determining the N-glycan composition of a serum sample obtained from an individual or patient at a time following the start of the an anti-diabetic therapy or treatment; and (b) comparing the N-glycan composition to the N-glycan composition of a serum sample obtained from the individual or patient at a time period before the start of the an anti-diabetic therapy or treatment, wherein a decrease in the amount of at least one N-glycan selected from the group consisting of high mannose N-glycan, hybrid N-glycans, complex N-glycans, or O-acetylated N-glycans in the N-glycan composition obtained from the serum sample obtained from the individual or patient at a time following the start of the anti-diabetic therapy or treatment indicates that the anti-diabetic therapy or treatment is efficacious.

In further embodiments of the above, the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight mass spectrometry (MALDI-TOF MS). In a further embodiment, the MALDI-TOF MS provides data that is analyzed by a computer to provide the N-glycan composition.

In particular embodiments, provided is a method of determining the efficacy of an an anti-diabetic therapy or treatment comprising (a) providing a first serum sample obtained from an individual or patient at a time before the start of an anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a difference between the first and second profiles indicates that the anti-diabetic therapy or treatment is efficacious.

In particular embodiments, provided is a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one high mannose N-glycan in the second profile compared to the first profile indicates that the anti-diabetic therapy or treatment is efficacious.

In particular embodiments, provided is a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one hybrid N-glycan in the second profile compared to the first profile indicates that the anti-diabetic therapy or treatment is efficacious.

In particular embodiments, provided is a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one complex N-glycan in the second profile compared to the first profile indicates that the anti-diabetic therapy or treatment is efficacious.

In particular embodiments, provided is a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one O-acetylated N-glycan in the second profile compared to the first profile indicates that the anti-diabetic therapy or treatment is efficacious.

In particular embodiments, provided is a method of determining the efficacy of an anti-diabetic therapy or treatment comprising (a) providing a first serum sample obtained from an individual or patient at a time before the start of the anti-diabetic therapy or treatment and a second serum sample obtained from the individual from a time following the start of the anti-diabetic therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one high mannose N-glycan, hybrid N-glycan, complex N-glycan, or O-acetylated N-glycan in the second profile compared to the first profile indicates that the anti-diabetic therapy or treatment is efficacious.

In further embodiments of the above, the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight (MALDI-TOF) to provide the N-linked glycosylation profile. In a further embodiment, the MALDI-TOF provides data that is analyzed by a computer to provide the N-linked glycosylation profile.

In particular embodiments of the above, the high mannose N-glycans are selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

In particular embodiments of the above, the hybrid N-glycans are selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments of the above, the O-acetylated (O-Ac) N-glycans are selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments of the above, the complex N-glycan is Sia₂Gal₂GlcNAc₂Man₃GlcNAc₂ (540020), wherein Sia is Neu5Ac or Neu5Gc.

In a further embodiments of the above, the N-glycan composition obtained from the individual or patient at a time following the start of the therapy or treatment comprises an increase in one or more fucosylated N-glycans. In particular aspects, the fucosylated N-glycans are selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc.

In further embodiments of the above, one or more serum samples were obtained from the individual or patient from a time selected from 3, 4, 5, 6, 7, 8, 9, 10, 11, 12, 13, 14, 15, 16, 17, 18, 19, 20, and 21 days following the start of the therapy or treatment. In particular aspects, serum samples were obtained from the individual or patient from a time selected from about day 7 and/or about day 14 following the start of the therapy or treatment.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (c) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; (c) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (d) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (c) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises an increase in one or more fucosylated N-glycans. In a further aspect, the change in the N-linked glycosylation pattern or profile of total serum proteins over time in an individual or patient undergoing an anti-diabetic therapy or treatment that is observed comprises an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) a decrease in one or more N-glycans selected from the group consisting of Man₉GlcNAc₂ (920000), MangGlcNAc₂ (820000), Man₇GlcNAc₂ (720000), Man₆GlcNAc₂ (620000), and Man₅GlcNAc₂ (520000).

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc and (b) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and (c) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); and (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (d) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (d) a decrease in a complex N-glycan such as Sia₂Gal₂GlcNAc₂Man₃GlcNAc₂(540020), wherein Sia is Neu5Ac or Neu5Gc; and (e) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) a decrease in one or more N-glycans selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000); (c) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (d) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (c) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (c) a decrease in a complex N-glycan such as Sia₂Gal₂GlcNAc₂Man₃GlcNAc₂ (540020), wherein Sia is Neu5Ac or Neu5Gc.

In particular embodiments, the change in the N-linked glycosylation pattern or profile of total serum proteins over time in an individual or patient undergoing an anti-diabetic therapy or treatment that is observed comprises (a) an increase in one or more N-glycans selected from the group consisting of Sia₃Gal₃GlcNAc₃Man₃GlcNAc₂(Fuc) (651030), Sia₃Gal₃GlcNAc₃Man₃GlcNAc₂(Fuc)(1 O-Ac) (651031), and Sia₄Gal₄GlcNAc₄Man₃GlcNAc₂(Fuc) (761040), wherein Sia is Neu5Ac or Neu5Gc; (b) decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; (c) a decrease in a complex N-glycan such as Sia₂Gal₂GlcNAc₂Man₃GlcNAc₂ (540020), wherein Sia is Neu5Ac or Neu5Gc; and (d) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments of the above, the anti-diabetic therapy or treatment comprises one or more insulin sensitizers. Insulin sensitizers include but are not limited to biguanides and thiazolidinediones wherein the biguanides include but are not limited to metformin, phenformin, and buformin and the thiazolidinediones include but are not limited to rosiglitazone, pioglitazone, and troglitazone.

In particular embodiments of the above, the anti-diabetic therapy or treatment comprises one or more insulin secretagogues. Insulin secretagogues include but are not limited to sulfonylureas and non-sulfonylureas wherein the sulfonylureas include but are not limited to tolbutamide, acetohexamide, tolazamide, chlorpropamide, glipizide, glyburide, glimepiride, and gliclazide and the non-sulfonylurease include but are not limited to metglitinides such as repaglinide and nateglinide.

In particular embodiments of the above, the anti-diabetic therapy or treatment comprises one or more the alpha-glucosidase inhibitors. Alpha-glucosidase inhibitors include but are not limited to miglitol and acarbose.

In particular embodiments of the above, the anti-diabetic therapy or treatment comprises one or more incretin or incretin mimetics. Incretin or incretin memetics include but are not limited to GLP 1 receptor agonists such as GLP 1, oxyntomodulin, exenatide, liraglutide, taspoglutide, and glucagon analogs that have GLP1 receptor agonist activity. DPP4 inhibitors include but are not limited to vildagliptin, sitagliptin, saxagliptin, and linagliptin.

In particular embodiments, provided is a method of determining the efficacy of an insulin therapy or treatment comprising (a) determining the N-glycan composition of a serum sample obtained from an individual or patient at a time following the start of the insulin therapy or treatment; and (b) comparing the N-glycan composition to the N-glycan composition of a serum sample obtained from the individual or patient at a time period before the start of the insulin therapy or treatment, wherein a difference in the N-glycan composition indicates that the insulin therapy or treatment is efficacious.

In particular embodiments, provided is a method of determining the efficacy of an insulin therapy or treatment comprising (a) determining the N-glycan composition of a serum sample obtained from an individual or patient at a time following the start of the insulin therapy or treatment; and (b) comparing the N-glycan composition to the N-glycan composition of a serum sample obtained from the individual or patient at a time period before the start of the insulin therapy or treatment, wherein a decrease in the amount of at least one high mannose N-glycan in the N-glycan composition obtained from the serum sample obtained from the individual or patient at a time following the start of the insulin therapy or treatment indicates that the insulin therapy or treatment is efficacious.

In particular embodiments, provided is a method of determining the efficacy of an insulin therapy or treatment comprising (a) determining the N-glycan composition of a serum sample obtained from an individual or patient at a time following the start of the insulin therapy or treatment; and (b) comparing the N-glycan composition to the N-glycan composition of a serum sample obtained from the individual or patient at a time period before the start of the insulin therapy or treatment, wherein a decrease in the amount of at least one hybrid N-glycan in the N-glycan composition obtained from the serum sample obtained from the individual or patient at a time following the start of the insulin therapy or treatment indicates that the anti-diabetic therapy or treatment is efficacious.

In further embodiments of the above, the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight mass spectrometry (MALDI-TOF MS). In a further embodiment, the MALDI-TOF MS provides data that is analyzed by a computer to provide the N-glycan composition.

In particular embodiments, provided is a method of determining the efficacy of an insulin therapy or treatment comprising (a) providing a first serum sample obtained from an individual or patient at a time before the start of the insulin therapy or treatment and a second serum sample obtained from the individual from a time following the start of the insulin therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a difference between the first and second profiles indicates that the insulin therapy or treatment is efficacious.

In particular embodiments, provided is a method of determining the efficacy of an insulin therapy or treatment comprising (a) providing a first serum sample obtained from an individual or patient at a time before the start of the insulin therapy or treatment and a second serum sample obtained from the individual from a time following the start of the insulin therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one high mannose N-glycan in the second profile compared to the first profile indicates that the insulin therapy or treatment is efficacious.

In particular embodiments, provided is a method of determining the efficacy of an insulin therapy or treatment comprising (a) providing a first serum sample obtained from an individual or patient at a time before the start of the insulin therapy or treatment and a second serum sample obtained from the individual from a time following the start of the insulin therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one hybrid N-glycan in the second profile compared to the first profile indicates that the insulin therapy or treatment is efficacious.

In particular embodiments, provided is a method of determining the efficacy of an insulin therapy or treatment comprising (a) providing a first serum sample obtained from an individual or patient at a time before the start of the insulin therapy or treatment and a second serum sample obtained from the individual from a time following the start of the insulin therapy or treatment; (b) determining the N-glycan composition of the first serum sample to obtain a first N-linked glycosylation profile and determining the N-glycan composition of the second serum sample to obtain a second N-linked glycosylation profile; and (b) comparing the first and second profiles, wherein a decrease in the amount of at least one high mannose N-glycan or hybrid N-glycan in the second profile compared to the first profile indicates that the insulin therapy or treatment is efficacious.

In further embodiments of the above, the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight (MALDI-TOF) to provide the N-linked glycosylation profile. In a further embodiment, the MALDI-TOF provides data that is analyzed by a computer to provide the N-linked glycosylation profile.

In particular embodiments, the high mannose N-glycans are selected from the group consisting of Man₈GlcNAc₂ (820000), Man₇GlcNAc₂ (720000), Man₆GlcNAc₂ (620000), and Man₅GlcNAc₂ (520000).

In particular embodiments, the hybrid N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments, the change in the N-linked glycosylation pattern or profile of total serum proteins over time in an individual or patient undergoing an insulin therapy or treatment that is observed comprises (a) a decrease in one or more N-glycans selected from the group consisting of Man₈GlcNAc₂ (820000), Man₇GlcNAc₂ (720000), Man₆GlcNAc₂ (620000), and Man₅GlcNAc₂ (520000); and (b) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

In particular embodiments of the above, the insulin is a native human insulin or human insulin analog or derivative.

In further embodiments of the above, one or more serum samples were obtained from the individual or patient from a time selected from 3, 4, 5, 6, 7, 8, 9, 10, 11, 12, 13, 14, 15, 16, 17, 18, 19, 20, and 21 days following the start of the therapy or treatment. In particular aspects, serum samples were obtained from the individual or patient from a time selected from about day 7 and/or about day 14 following the start of the therapy or treatment.

### Example 1

The objective of Study 1 was to evaluate N-linked glycosylation changes in plasma proteins that precede and predict the decrease in glycated hemoglobin (HbA1c) associated with successful resolution of diabetes. Diabetic (db/db) mice were treated once daily with an oral dose of 10 mpk rosiglitazone or with vehicle. Samples included plasma from 20 db/db mice (ten vehicle and ten rosiglitazone) at each of seven time points: 3, 7, 10, 14, 21, 31, and 39 days. A baseline (Day 0) sample was not analyzed in the initial rosiglitazone study, but was included in the subsequent studies described in Examples 2 and 3.

FIG. 1 schematically shows the process for detecting the change in N-linked glycosylation of total serum proteins. GLYCANMAP Assay (a registered trademark of Ezose Sciences, Pine Brook, N.J.): A 10 μL aliquot of each plasma sample was spiked with internal standard (700 pmol) to aid in quantitation. The spiked aliquots were analyzed for N-linked glycans using GLYCANMAP methodology, which is based on the methods previously reported by Nishimura, Furukawa and Miura (Nishimura et al., Angew Chem. Int. Ed. Engl., 44: 91-96 (2004); Furukawa et al., Anal. Chem., 80: 1094-1101 (2008); Miura et al., Chem.-A Eur. J, 13: 4797-4804 (2007)). The aliquots were denatured and then digested with trypsin, followed by heat-inactivation. The mixture was then treated with PNGase F. After enzymatic release of N-glycans, aliquots were subjected to solid-phase processing using BLOTGLYCO beads (Sumitomo Bakelite Co., Ltd., Tokyo, Japan). Following capture on the beads, the sialic acid residues were methyl esterified. The N-glycans were simultaneously released from the beads and labeled, and then aliquots of the recovered materials were spotted onto a MALDI target plate. Steps from initial aliquoting to spotting on the MALDI plate were performed using the fully automated SWEETBLOT technology (System Instrument Co., Ltd.).

MALDI-TOF MS analysis was performed on an Ultraflex III mass spectrometer (Bruker Daltonics, Billerica, Mass.) in the positive-ion, reflector mode using a proprietary matrix composition. Each sample from the BLOTGLYCO processing step was spotted in quadruplicate, and spectra were obtained in an automated manner using the AutoXecute feature in flexControl software. Mass spectra were analyzed using a bioinformatics program. Because the chemical derivatization inherent in the GLYCANMAP technology can produce additional minor species with different mass from that of the parent N-glycan, the data analysis methodologies contain algorithms to correct for this. Some minor peaks can be difficult to interpret as true N-glycans due to either low signal-to-noise ratios or partial overlap with other peaks. Therefore, spectra were also visually inspected to eliminate false positives and negatives. N-glycan structures were assigned based on molecular weight and literature precedent. In some cases, additional isomeric structures may be formed, which may be resolved by additional MS-MS analysis.

### Data Analysis

N-glycan concentrations were compared between treatment groups using a variety of statistical tests. Rosiglitazone-treated and vehicle-treated db/db mice were compared across each time point using the Mann-Whitney test. N-glycans which yielded p-values <0.05 in this analysis were considered significant. N-glycan changes were then prioritized if they demonstrated 1) significant differences between treatment groups at multiple time points and 2) sustained or increasing differences between treatment groups over time.

### Results

The initial rosiglitazone study revealed statistically significant changes in 16 out of 52 individual N-glycans (Table 1). Twelve of the 16 candidate biomarkers yielded highly significant differences (p-values <0.001) after seven days of treatment, with some glycans exhibiting significant differences after only 3 days. By comparison, this level of statistical significance was not achieved for HbA1c until 21 days, suggesting that changes in glycosylation on the circulating glycoproteins can predict subsequent changes in the level of glycation in HbA1c by approximately two weeks in this model. Glycan biomarkers could be grouped into several categories based on their structure. High-mannose, hybrid, and O-acetylated glycans decreased with successful glycemic control whereas fucosylated glycans increased.

TABLE 1
Glycan Changes Associated with
Glycemic Control
(Rosiglitazone Studies)

| Glycan Category and | Direction of Change |
|---|---|
| Code | with Rosiglitazone |
| High Mannose | |
| 5 2 0 0 0 0 | Decreased |
| 6 2 0 0 0 0 | Decreased |
| 7 2 0 0 0 0 | Decreased |
| 8 2 0 0 0 0 | Decreased |
| 9 2 0 0 0 0 | Decreased |
| Fucosylated | |
| 6 5 1 0 3 0 | Increased |
| 6 5 1 0 3 1 | Increased |
| 7 6 1 0 4 0 | Increased |
| O-Acetylated | |
| 5 4 0 0 2 1 | Decreased |
| 5 4 0 0 2 2 | Decreased |
| 5 4 0 0 3 1 | Decreased |
| 5 4 0 0 3 2 | Decreased |
| Hybrid | |
| 4 3 0 0 1 0 | Decreased |
| 5 3 0 0 1 0 | Decreased |
| 6 3 0 0 1 0 | Decreased |
| Complex | |
| 5 4 0 0 2 0 | Decreased |

Several criteria were used in the initial rosiglitazone study to select the most promising markers. Statistical significance was evaluated based on the Mann-Whitney test and changes considered significant if the resulting p-value was less than 0.05. Statistically significant differences were then compared across all available time points and only N-glycans that demonstrated statistically significant differences at six of the seven time points and that exhibited changes that were sustained throughout the 39 day treatment period were selected. After the second rosiglitazone study (shown in Example 2), which focused on changes at Day 7, one glycan (530010) that had been excluded in Study 1 was re-evaluated. This N-glycan was significant at five of seven time points in Study 1 and exhibited statistically significant differences at Day 7 in both rosiglitazone studies, and was therefore added to the original list of candidate markers. Most of the N-glycans exhibiting statistically significant differences between treatment groups could be classified into structurally-defined categories as set forth below.

### High-Mannose Glycans

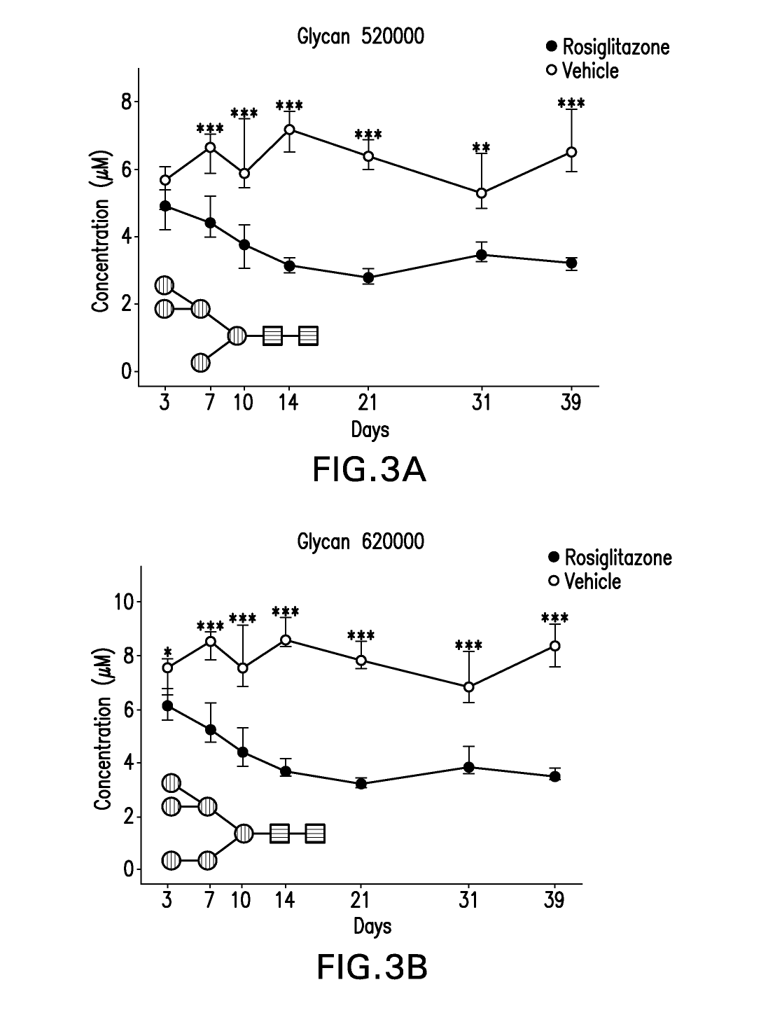

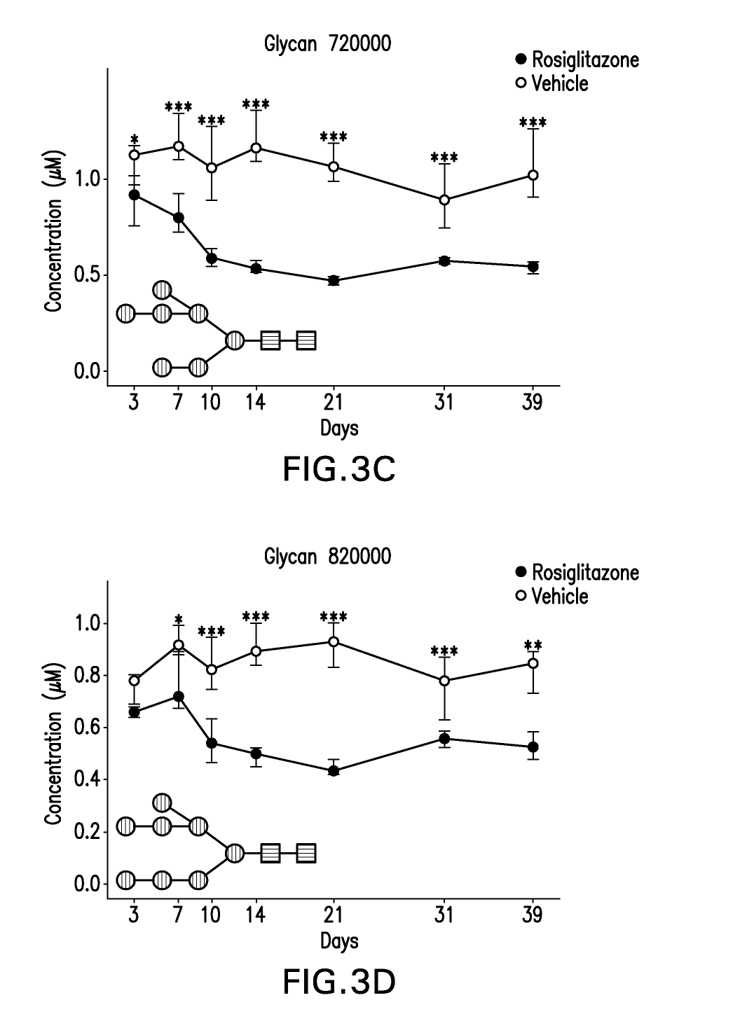

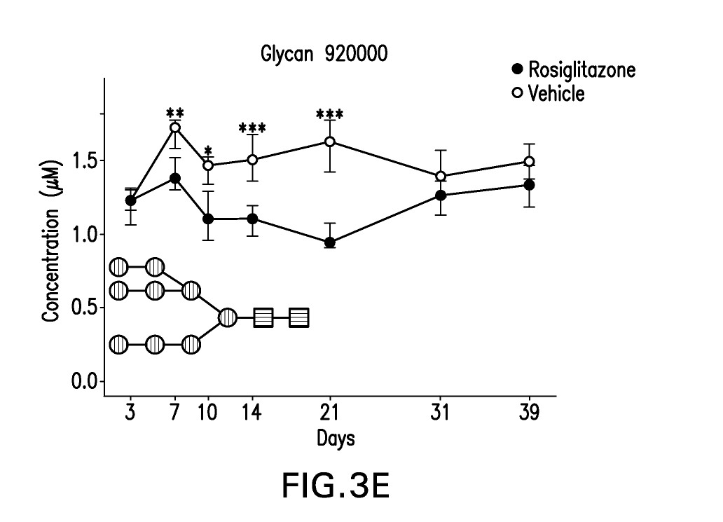

All five detected high-mannose N-glycans, including Man₅GlcNAc₂, Man₆GlcNAc₂, Man₇GlcNAc₂, MangGlcNAc₂, and Man₉GlcNAc₂ (glycan codes 520000, 620000, 720000, 820000, and 920000, respectively) were lower in rosiglitazone-treated db/db mice compared to vehicle-treated db/db mice (FIG. 3A-3E). Changes in all five high-mannose N-glycans were significant at Day 7, with two N-glycans (Man₆GlcNAc₂ and Man₇GlcNAc₂) exhibiting statistically significant differences between treatment groups at Day 3.

### Fucosylated Glycans

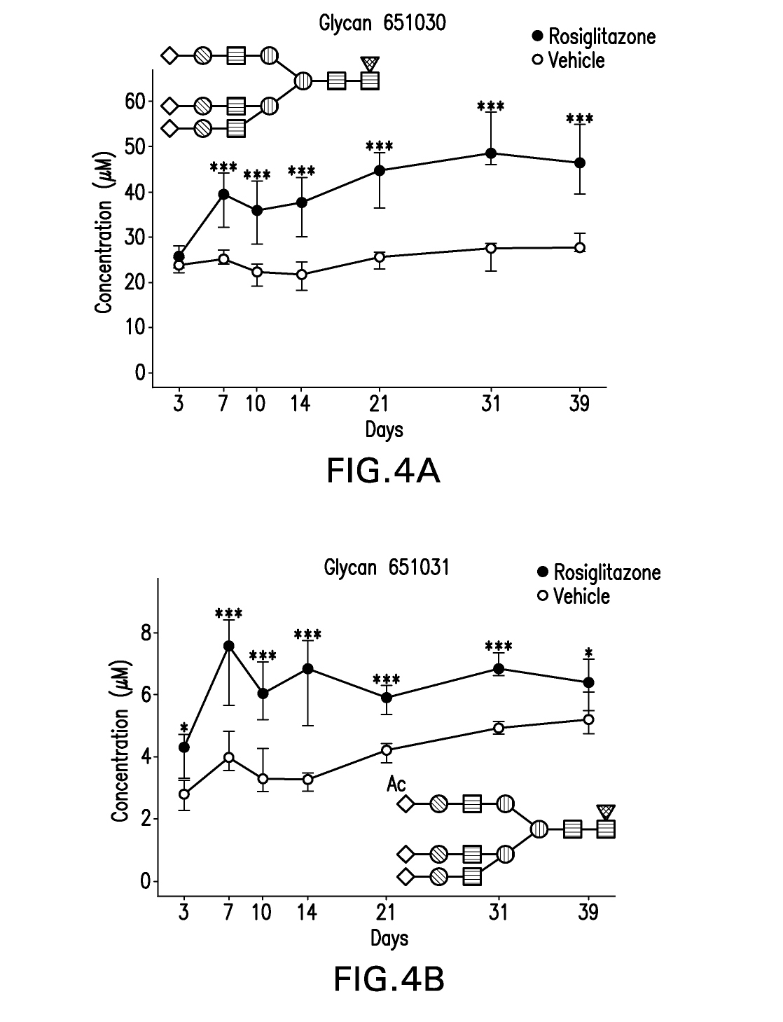

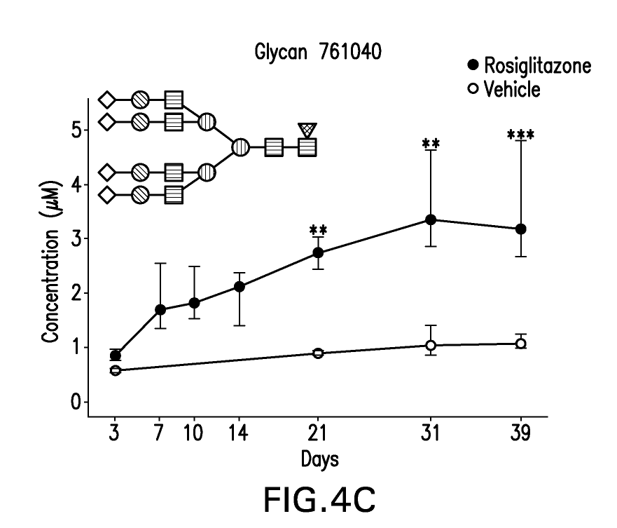

Several fucosylated glycans, including glycans 651030, 651031, and 761040 exhibited significantly higher levels in rosiglitazone-treated db/db mice compared to vehicle controls (FIG. 4A-4C). Glycan 651030 and 651031 exhibited highly significant differences (p<0.001) at 7 days which were sustained at all subsequent time points. Glycan 651031 also exhibited significant differences at Day 3. A third glycan (761040) showed a similar trend but was lower abundance, making it difficult to quantitate in some samples.

### O-Acetylated Glycans

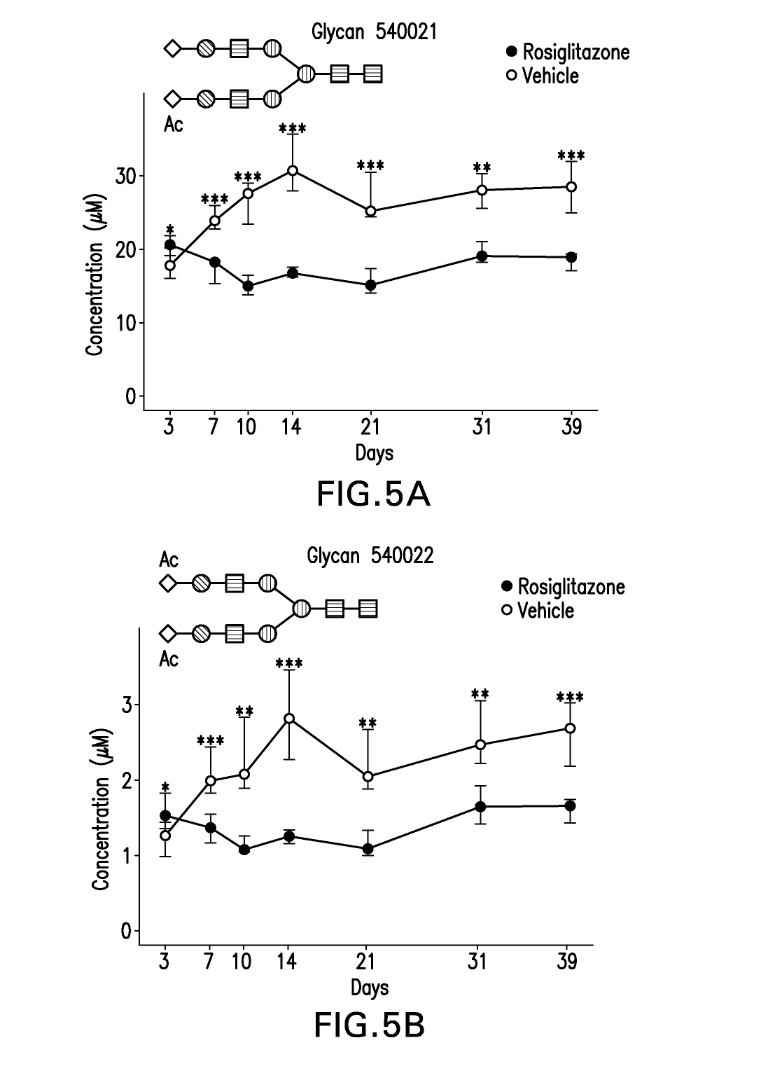

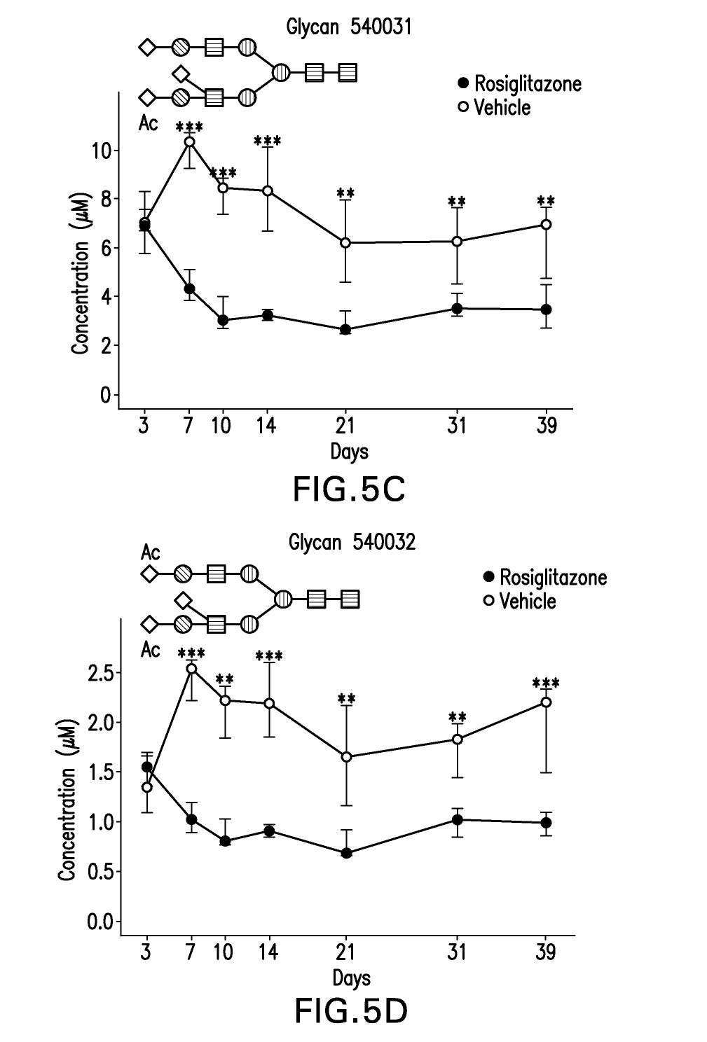

Acetylation of sialic acids in N-glycans is common in mice but is less common in humans. While acetylation of sialic acids has been reported in humans in cancerous cells, the presence and/or extent of O-acetylation in diabetes is unknown. Several O-acetylated N-glycans exhibited statistically significant differences between treatment groups. Four O-acetylated N-glycans, with glycan codes of 540021, 540022, 540031, and 540032 (FIG. 5A-5D) exhibited significant lower levels (p<0.001) in rosiglitazone-treated db/db mice as early as seven days, which were sustained through the rest of the study. Glycans 540021 and 540022 showed significant differences as early as Day 3.

### Changes in Hybrid Glycans

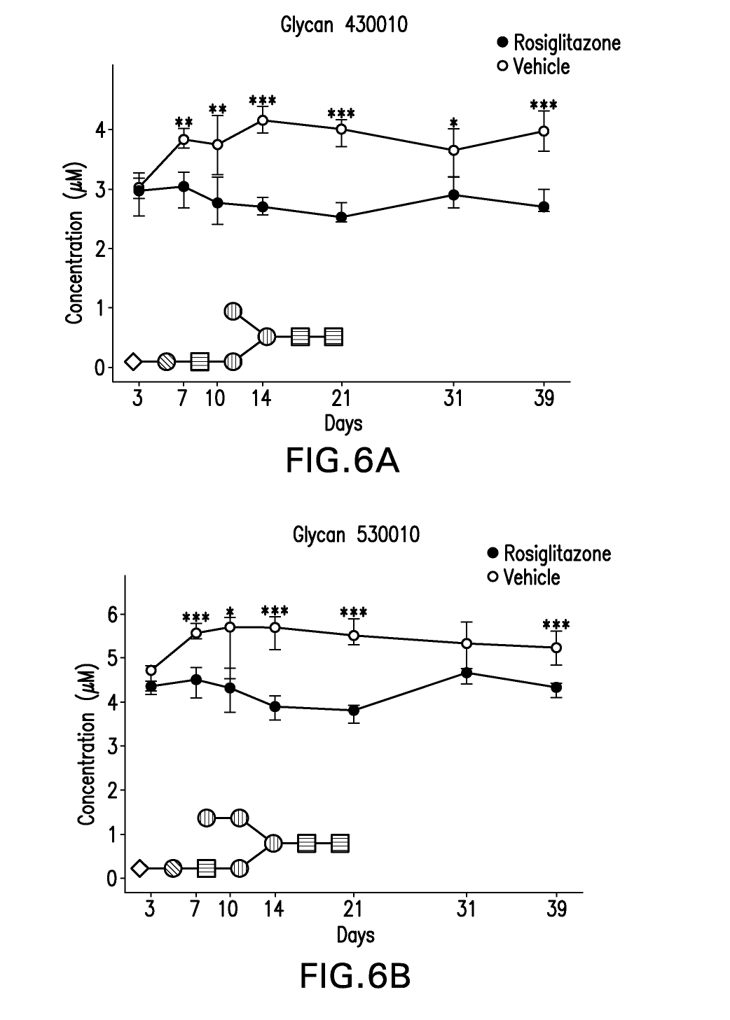

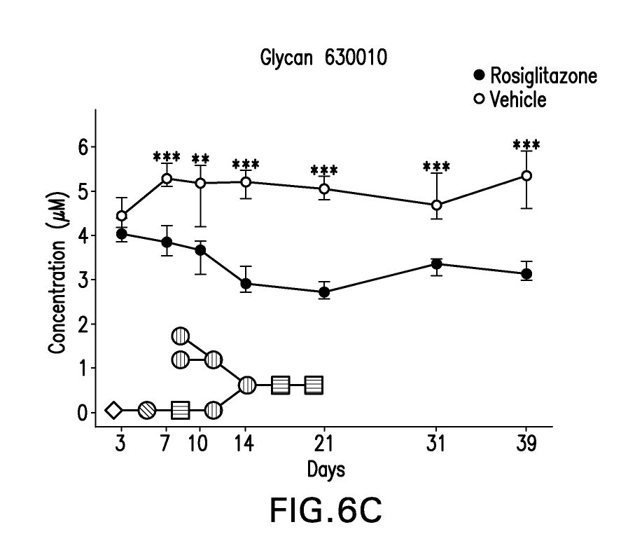

Three hybrid glycans (430010, 530010, and 630010) exhibited lower levels in rosiglitazone-treated db/db mice compared to the vehicle controls in the first rosiglitazone study (FIG. 6A-6C).

### Changes in Complex Glycans

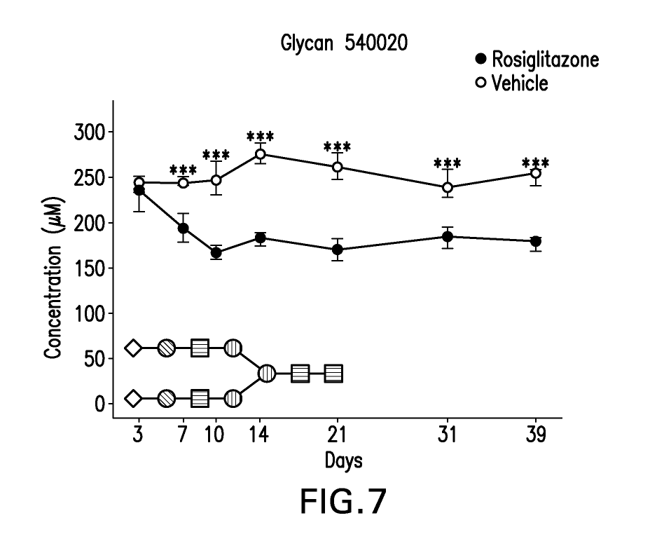

Glycan 540020, a complex glycan, also exhibited highly significant differences in rosiglitazone-treated mice compared to vehicle. In the first rosiglitazone study, Glycan 540020 exhibited a significant decrease in rosiglitazone-treated mice at Day 7 (p<0.001) which was sustained at subsequent time points (FIG. 7).

### Example 2

A second study, Study 2, was designed to verify and further characterize biomarker candidates observed in a previous study (Study 1) in a separate in vivo study, focusing on the changes that occur in the first 7 days. Study 2 included plasma from ten db/db mice at baseline (0 days, a time point that was not included in Study 1) and plasma from 20 db/db mice (ten vehicle and ten rosiglitazone) at seven days. Statistical significance of differences between treatment groups and over time was evaluated using the Student's t-test.

### High-Mannose Glycans

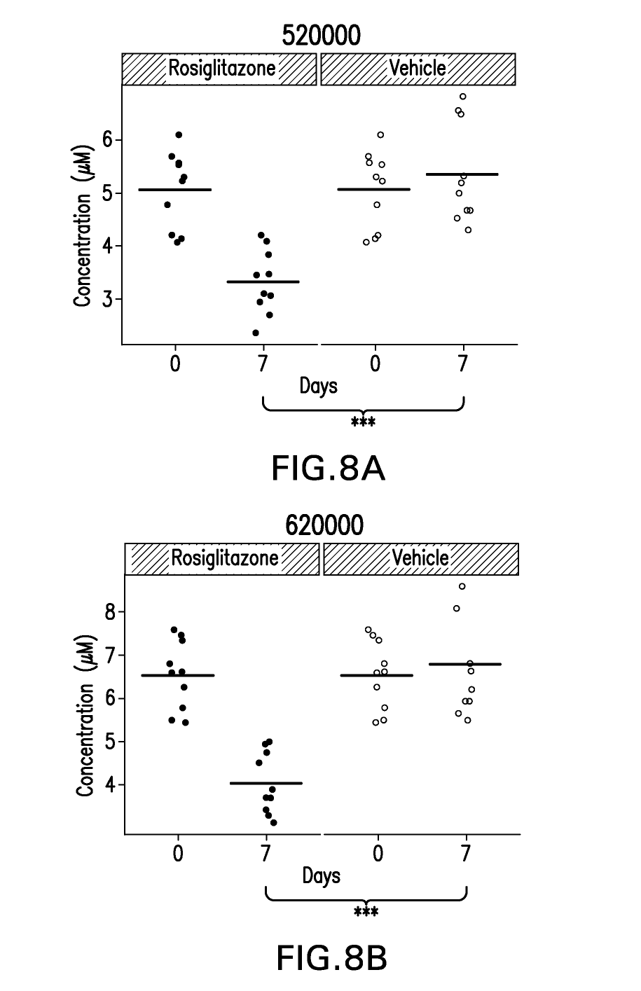

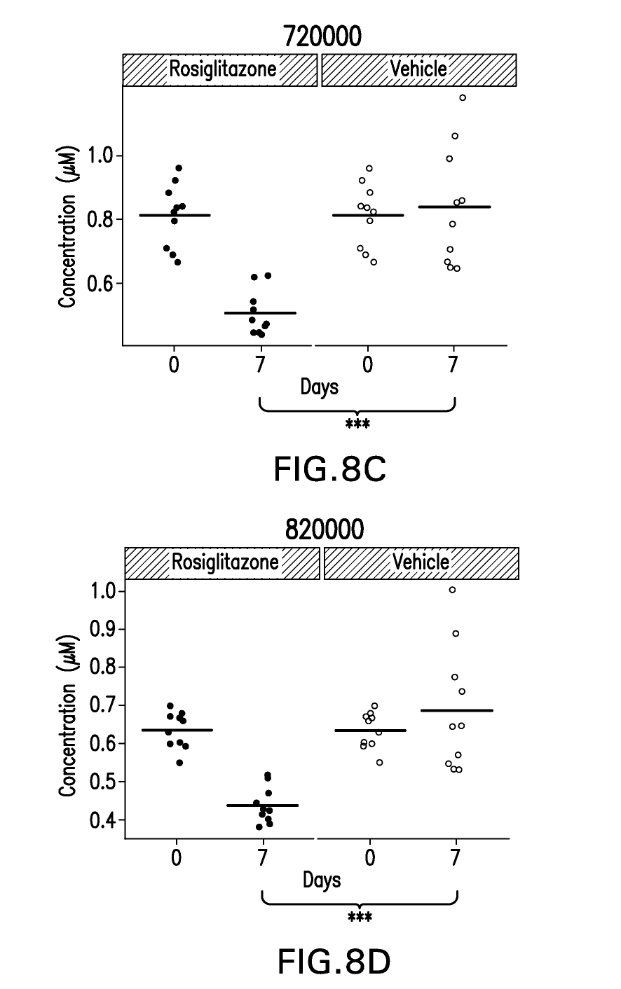

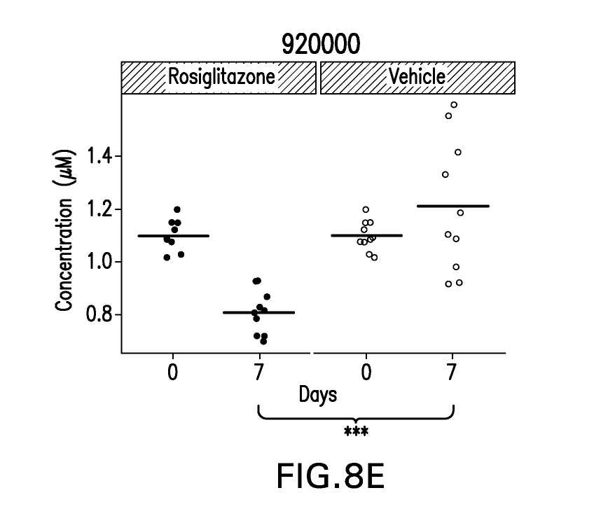

All five detected high-mannose N-glycans, including Man₅GlcNAc₂, Man₆GlcNAc₂, Man₇GlcNAc₂, Man₈GlcNAc₂, and Man₉GlcNAc₂ (glycan codes 520000, 620000, 720000, 820000, and 920000, respectively) were significantly lower (p<0.001) in rosiglitazone-treated db/db mice compared to vehicle-treated db/db mice (FIG. 8A-8E) as was observed in the first rosiglitazone study (Example 1).

### Fucosylated Glycans

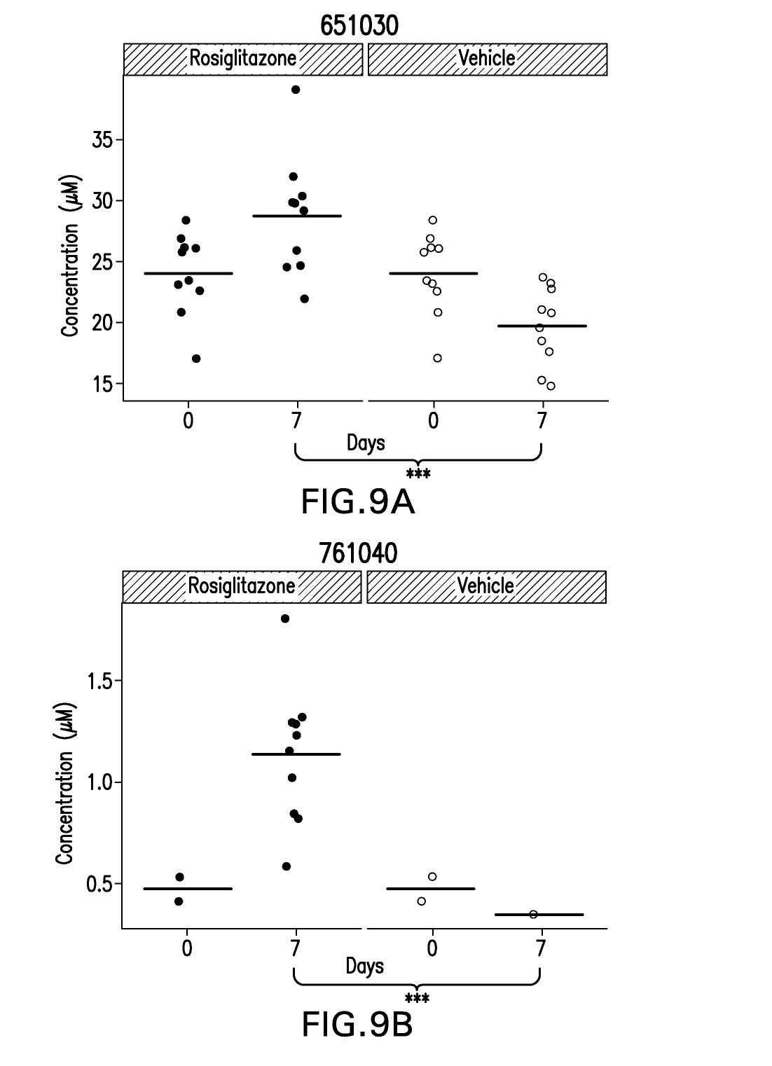

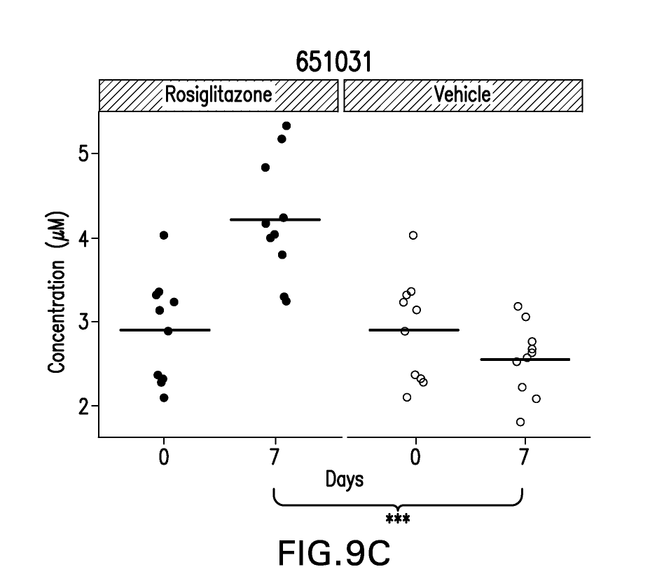

Several fucosylated glycans, including glycans 651030, 651031, and 761040 exhibited significantly higher levels in rosiglitazone-treated db/db mice compared to vehicle controls (FIG. 9A-9C) as was observed in the first rosiglitazone study.

### O-Acetylated Glycans

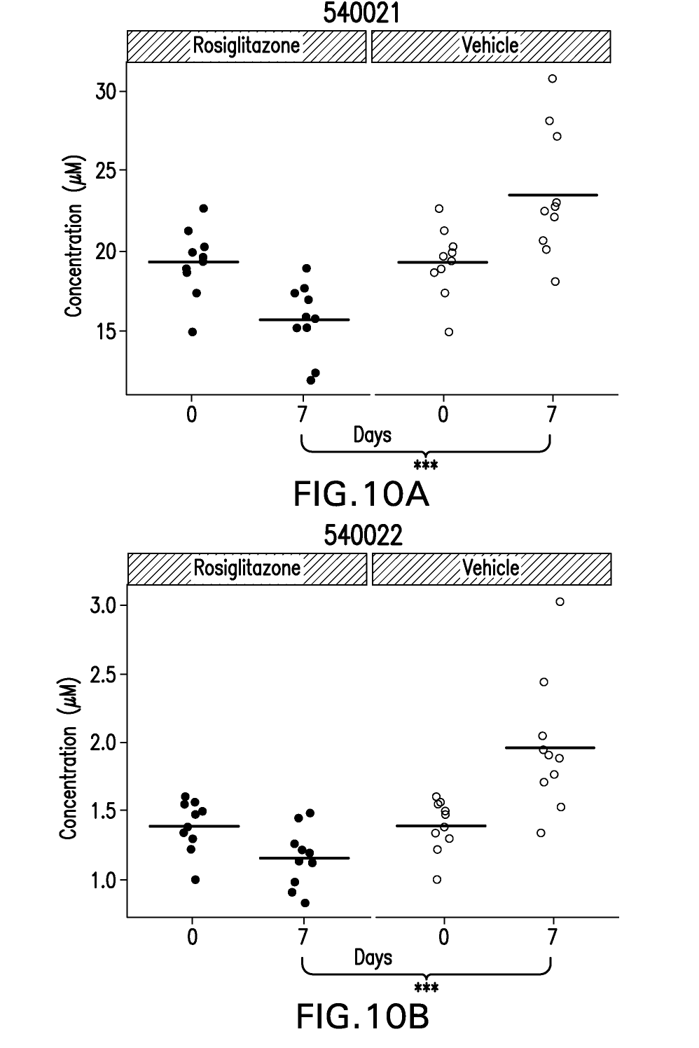

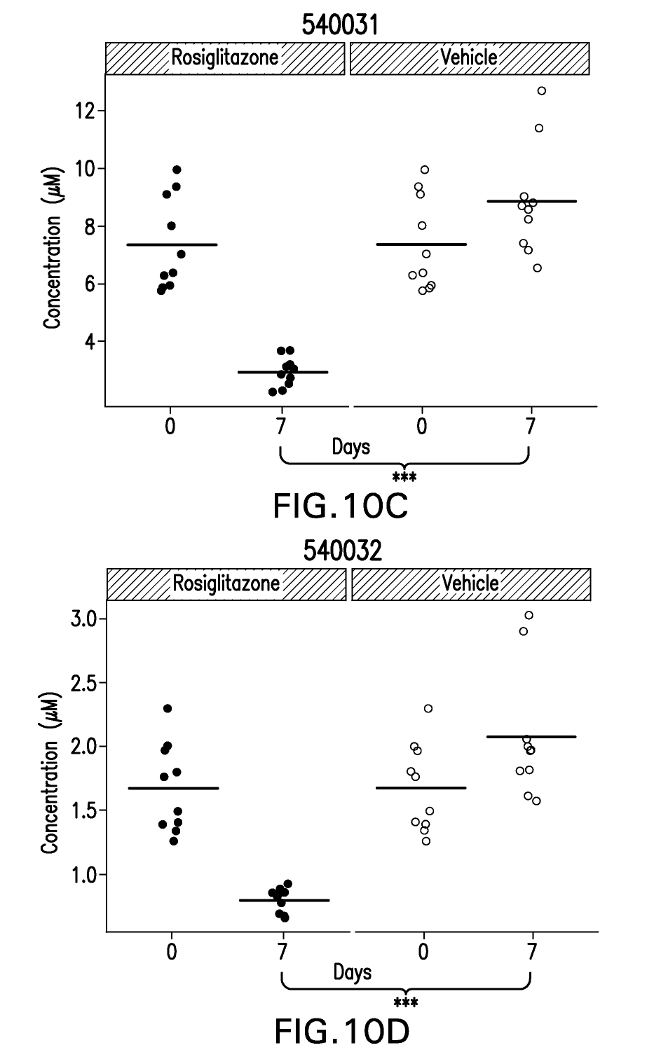

The four O-acetylated N-glycans, with glycan codes of 540021, 540022, 540031, and 540032 (FIG. 10A-10D) exhibited significant lower levels (p<0.001) in rosiglitazone-treated db/db mice at seven days, as was observed in the first rosiglitazone study.

### Changes in Hybrid Glycans

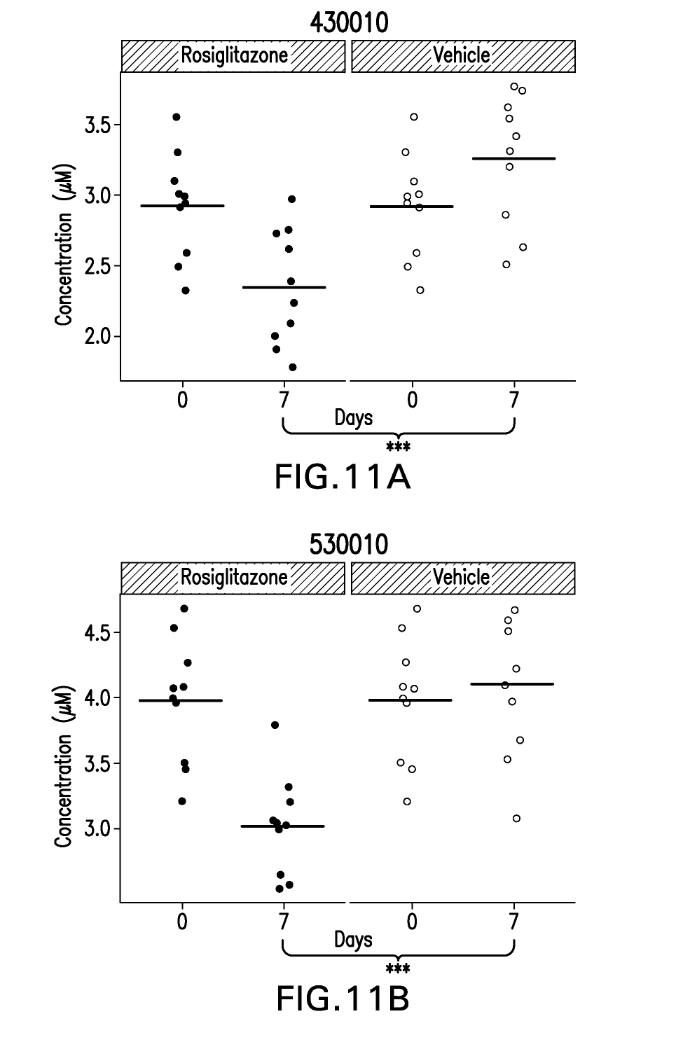

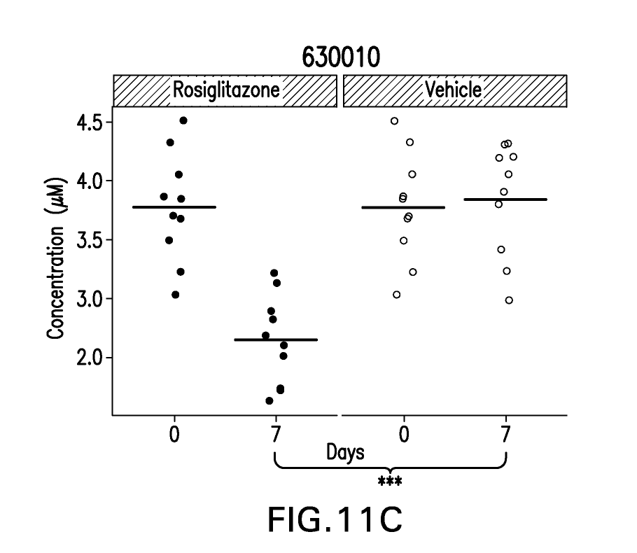

Three hybrid glycans (430010, 530010, and 630010) exhibited significantly lower levels (p<0.001) in rosiglitazone-treated db/db mice compared to the vehicle controls in the second rosiglitazone study (FIG. 11A-11C) as was observed in the first rosiglitazone study.

### Changes in Complex Glycans

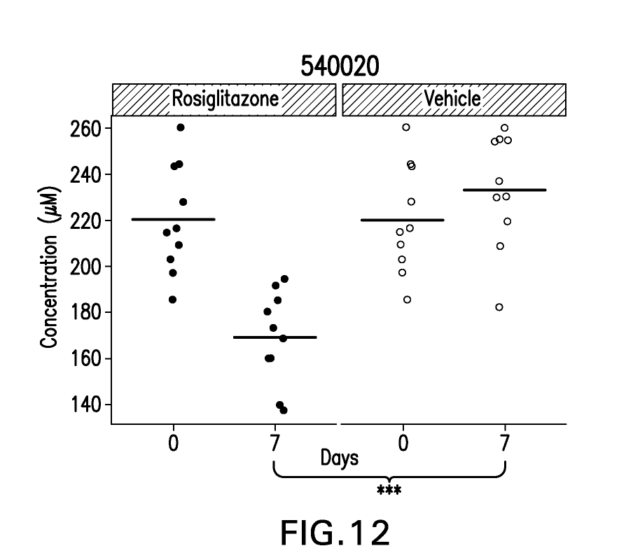

Complex glycan 540020 exhibited a significant decrease in rosiglitazone-treated mice at Day 7 (p<0.001) (FIG. 12), as was observed in the first rosiglitazone study.

### Example 3

The objective of this study was to evaluate the performance of candidate biomarkers discovered using rosiglitazone in mice treated with a diabetes drug with a different mechanism of action. The 16 candidate markers that were identified in Example 1 were evaluated in db/db mice treated with insulin detemir and vehicle.

In this Example, plasma samples were analyzed from ten db/db mice at baseline (0 days), and 20 db/db mice (ten vehicle and ten insulin detemir) at 7, 14, and 21 days. Sample preparation and analysis followed the protocol described in Example 1.

Data Analysis: For this study, the analysis was extended to evaluate changes in all detected N-glycans in order to reveal any novel N-glycan changes that might be specific to insulin detemir. The extended analysis was not performed in Study 2, which included only baseline and Day 7 time points since N-glycan changes at Day 7 could not be evaluated to determine if they are sustained over time. Drug-treated and vehicle-treated db/db mice were compared across each time point using the Student's t-test. To evaluate time-dependent changes, each time point within a treatment group was compared to its corresponding Day 0 group. N-glycans which yielded p-values <0.05 in this analysis were considered significant.

### Analysis of Changes in Individual N-Glycans

The concentrations of individual N-glycans in insulin detemir- and vehicle-treated db/db mice were compared at each time point using the Student's t-test. Differences were considered statistically significant if they demonstrated a p<0.05. Six of the sixteen N-glycans selected as candidate markers in Example 1 also exhibited statistically significant differences between insulin detemir- and vehicle-treated db/db mice (Table 2). Time-dependence was also evaluated for each of the candidate markers by comparing each time point to baseline.

TABLE 2
Glycan Changes Associated
with Glycemic Control
(Insulin Detemir Study)

| | Direction of Change |
|---|---|
| Glycan Category | with Insulin |
| and Code | Detemir |
| High Mannose | |
| 5 2 0 0 0 0 | Decreased |
| 6 2 0 0 0 0 | Decreased |
| 7 2 0 0 0 0 | Decreased |
| 8 2 0 0 0 0 | Decreased |
| Hybrid | |
| 4 3 0 0 1 0 | Decreased |
| 5 3 0 0 1 0 | Decreased |
| 6 3 0 0 1 0 | Decreased |

### High-Mannose N-Glycans

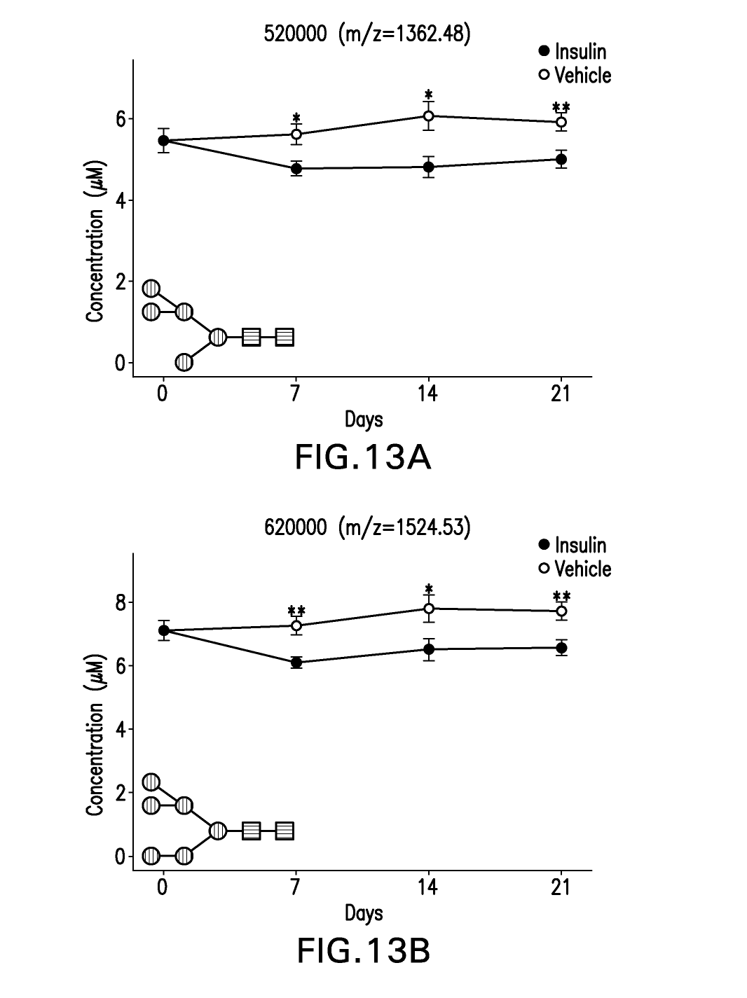

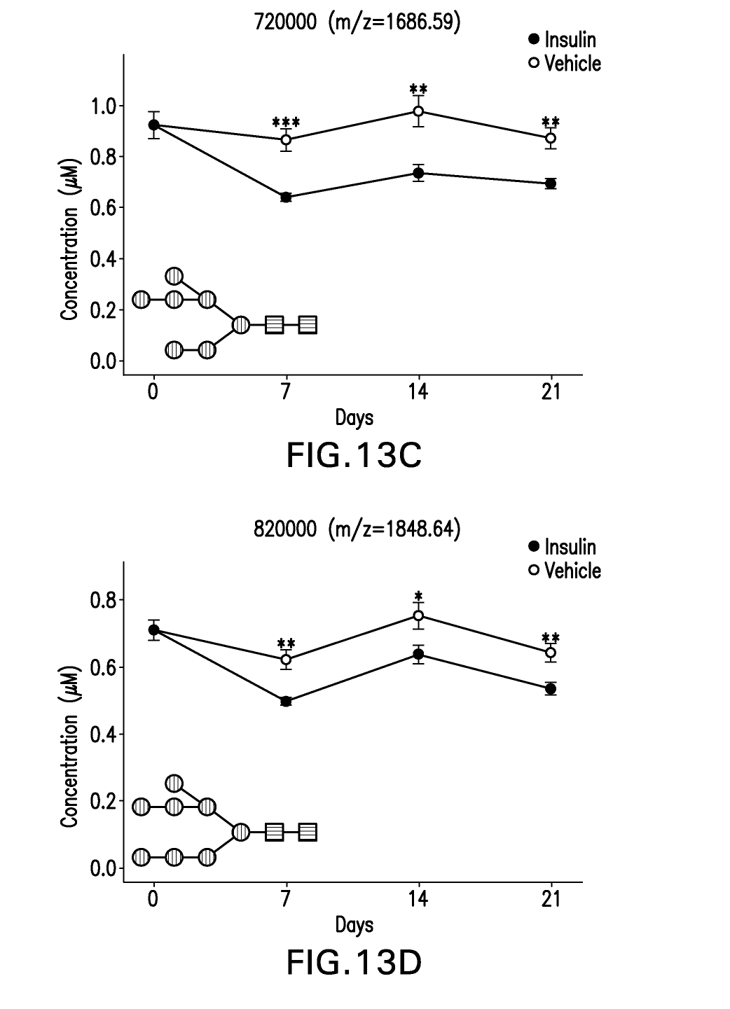

In the rosiglitazone studies, rosiglitazone-treated db/db mice exhibited a significant and sustained decrease in five high mannose N-glycans (Man₅GlcNAc₂, Man₆GlcNAc₂, Man₇GlcNAc₂, Man₈GlcNAc₂, and Man₉GlcNAc₂) as early as Day 7. While the insulin-induced changes were lower in magnitude than with rosiglitazone, insulin detemir-treated db/db mice also demonstrated lower levels of four of the five high mannose N-glycans, Man₅GlcNAc₂, Man₆GlcNAc₂, Man₇GlcNAc₂, and Man₈GlcNAc₂ (FIGS. 13A-13D). The differences were significant at Day 7 and remained significant at Day 14 and 21.

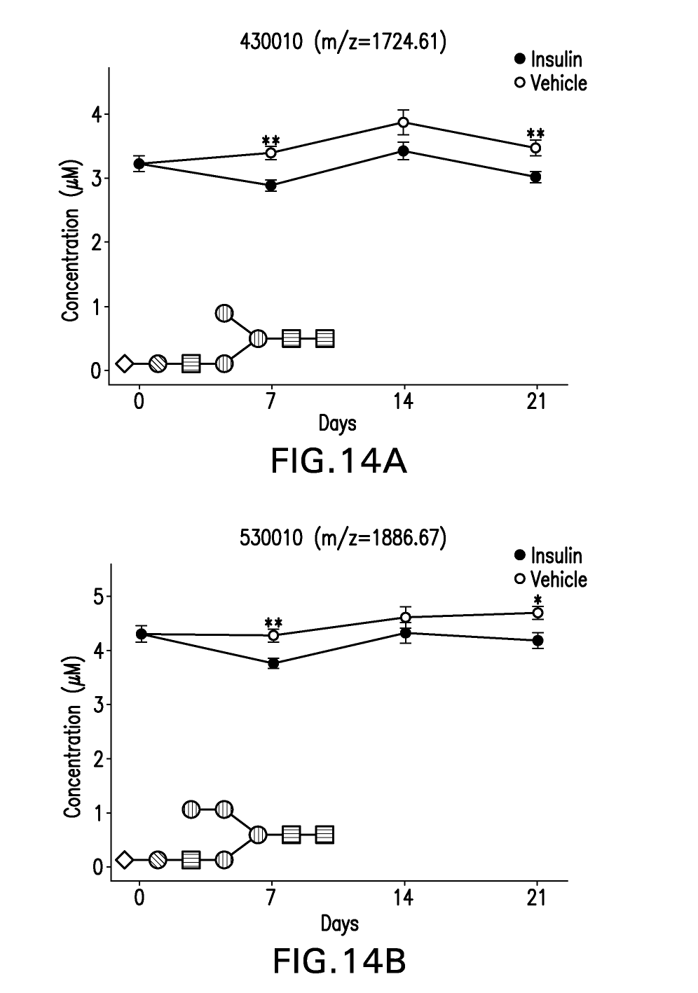

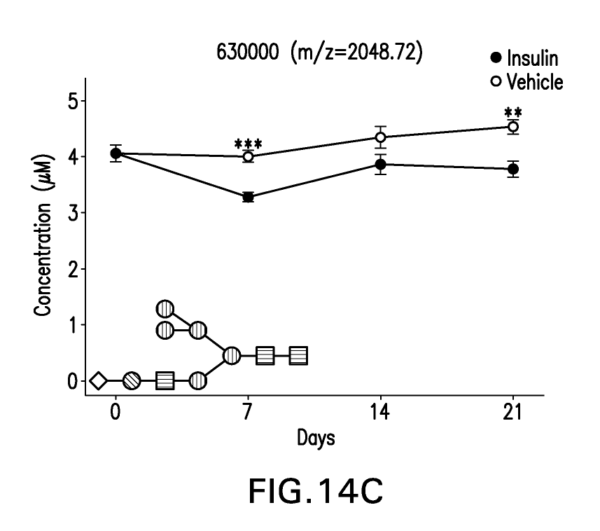

Three hybrid glycans, 430010, 530010, and 630010 demonstrated statistically significant differences between insulin detemir-treated db/db mice and their vehicle-treated controls (FIGS. 14A-14C). These glycans were also lower in rosiglitazone-treated mice in Studies 1 and 2. All three hybrid glycans showed significant decreases in insulin detemir-treated db/db mice as early as Day 7.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method of determining the efficacy of an anti-diabetic therapy or treatment comprising:
(a) determining the N-glycan composition of a serum sample obtained from an individual or patient at a time following the start of the anti-diabetic therapy or treatment; and
(b) comparing the N-glycan composition of the serum sample to the N-glycan composition of a serum sample obtained from the individual or patient at a time before the start of the anti-diabetic therapy or treatment,
wherein a difference between the N-glycan composition of the serum sample obtained from the individual or patient at a time following the start of the anti-diabetic therapy or treatment and the N-glycan composition of the serum sample obtained from the individual or patient at a time before the start of the anti-diabetic therapy or treatment indicates that the anti-diabetic therapy or treatment is efficacious.

2. The method of claim 1, wherein the difference in the N-glycan composition is a decrease in the amount of at least one high mannose N-glycan, hybrid N-glycan, complex N-glycan, or O-acetylated N-glycan in the N-glycan composition of the serum sample obtained from the serum sample obtained from the individual or patient at a time following the start of the anti-diabetic therapy or treatment compared to the amount of the corresponding N-glycan in the serum sample obtained from the individual or patient before the start of the anti-diabetic therapy or treatment.

3. The method of claim 1, wherein the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight (MALDI-TOF).

4. A method of determining the efficacy of an anti-diabetic therapy or treatment comprising:
(a) providing a first serum sample obtained from an individual or patient at a time before the start of the anti-diabetic therapy or treatment and a second serum sample obtained from the individual from a time following the start of the anti-diabetic therapy or treatment;
(b) determining the N-glycan composition of the first serum sample to obtain a first N-linked glycosylation profile and determining the N-glycan composition of the second serum sample to obtain a second N-linked glycosylation profile; and
(c) comparing the first and second N-linked glycosylation profiles, wherein a difference between the first and second N-linked glycosylation profiles indicates that the anti-diabetic therapy or treatment is efficacious.

5. The method of claim 4, wherein a decrease in the amount of at least one high mannose N-glycan, hybrid N-glycan, complex N-glycan, or O-acetylated N-glycan in the second N-linked glycosylation profile compared to the amount of the corresponding N-glycan in the first N-linked glycosylation profile indicates that the anti-diabetic therapy or treatment is efficacious.

6. The method of claim 4, wherein the N-glycan composition is determined by separating the N-glycans from the proteins in the serum sample to provide a composition of N-glycans and determining the relative amounts of N-glycans in the composition by Matrix Adsorption Laser Desorption/Ionization-Time-Of-Flight (MALDI-TOF).

7. The method of claim 5, wherein the high mannose N-glycan is Man₉GlcNAc₂ (920000), Man₈GlcNAc₂ (820000), Man₇GlcNAc₂ (720000), Man₆GlcNAc₂ (620000), or Man₅GlcNAc₂ (520000).

8. The method of claim 5, wherein the hybrid N-glycans is SiaGalGlcNAcMan₃GlcNAc₂ (430010), SiaGalGlcNAcMan₄GlcNAc₂ (530010), or SiaGalGlcNAcMan₅GlcNAc₂ (630010), wherein Sia is Neu5Ac or Neu5Gc.

9. The method of claim 5, wherein the O-acetylated (O-Ac) N-glycan is Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), or Sia₃Gal₂GlcNAc₂Man₃GlcNAc₂(2 O-Ac) (540032), wherein Sia is Neu5Ac or Neu5Gc.

10. The method of claim 5, wherein the complex N-glycan is Sia₂Gal₂GlcNAc₂Man₃GlcNAc₂ (540020), wherein Sia is Neu5Ac or Neu5Gc.

11. The method of claim 5, wherein the N-glycan composition obtained from the individual or patient at a time following the start of the therapy or treatment comprises an increase in the amount of one or more fucosylated N-glycans compared to the amount of the corresponding fucosylated N-glycan in the N-glycan composition obtained from the individual or patient before the start of the anti-diabetic therapy or treatment.

12. The method of claim 11, wherein the fucosylated N-glycan is Sia₃Gal₃GlcNAc₃Man₃GlcNAc₂(Fuc) (651030), Sia₃Gal₃GlcNAc₃Man₃GlcNAc₂(Fuc)(1 O-Ac) (651031), or Sia₄Gal₄GlcNAc₄Man₃GlcNAc₂(Fuc) (761040), wherein Sia is Neu5Ac or Neu5Gc.

13. The method of claim 4, wherein the difference in the N-linked glycosylation profile comprises (i) a decrease in one or more N-glycans selected from the group consisting of MangGlcNAc₂ (920000), Man₈GlcNAc₂ (820000), Man₇GlcNAc₂ (720000), Man₆GlcNAc₂ (620000), and Man₅GlcNAc₂ (520000); and (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

14. The method of claim 4, wherein the difference in the N-linked glycosylation profile comprises (i) a decrease in one or more N-glycans selected from the group consisting of Man₉GlcNAc₂ (920000), MangGlcNAc₂ (820000), Man₇GlcNAc₂ (720000), Man₆GlcNAc₂ (620000), and Man₅GlcNAc₂ (520000); (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc.

15. The method of claim 4, wherein the difference in the N-linked glycosylation profile comprises (i) a decrease in one or more N-glycans selected from the group consisting of Man₉GlcNAc₂ (920000), MangGlcNAc₂ (820000), Man₇GlcNAc₂ (720000), Man₆GlcNAc₂ (620000), and Man₅GlcNAc₂ (520000); (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; (iii) a decrease in one or more N-glycans selected from the group consisting of Sia₂Gal₂GlcNAc₂Man₃GlcNAc₂(1 O-Ac) (540021), Sia₂Gal₂GlcNAc₂Man₃GlcNAc₂(2 O-Ac) (540022), Sia₃Gal₂GlcNAc₂Man₃GlcNAc₂(1 O-Ac) (540031), and Sia₃Gal₂GlcNAc₂Man₃GlcNAc₂(2 O-Ac) (540032), wherein Sia is Neu5Ac or Neu5Gc; and (iv) a decrease in a complex N-glycan such as Sia₂Gal₂GlcNAc₂Man₃GlcNAc₂ (540020), wherein Sia is Neu5Ac or Neu5Gc.

16. The method of claim 4, wherein the difference in the N-linked glycosylation profile comprises (i) a decrease in one or more N-glycans selected from the group consisting of MangGlcNAc₂ (920000), Man₈GlcNAc₂ (820000), Man₇GlcNAc₂ (720000), Man₆GlcNAc₂ (620000), and Man₅GlcNAc₂ (520000); (ii) a decrease one or more N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; and (iii) a decrease in a complex N-glycan such as Sia₂Gal₂GlcNAc₂Man₃GlcNAc₂ (540020), wherein Sia is Neu5Ac or Neu5Gc.

17. The method of claim 4, wherein the one or more serum samples were obtained from the individual or patient from a time selected from 3, 4, 5, 6, 7, 8, 9, 10, 11, 12, 13, 14, 15, 16, 17, 18, 19, 20, and 21 days following the start of the therapy or treatment.

18. The method of claim 4, wherein the anti-diabetic therapy or treatment comprises insulin, an insulin sensitizer, insulin secretagogue, alpha-glucosidase inhibitor, incretin or incretin mimetic, dipetidyl peptidase 4 (DPP4) inhibitor, amylin or amylin analog, or GLP-1 receptor agonist.

19-21. (canceled)
